SEQUENCE LISTING (143 sequences)

SEQ ID NO: 1 - DNA, 17 bp, Artificial sequence
  Synthetic oligonucleotide
tcttttttttttttttt

SEQ ID NO: 2 - DNA, 17 bp, Artificial sequence
  Synthetic oligonucleotide
tcttttttttcgttttt

SEQ ID NO: 3 - DNA, 17 bp, Artificial sequence
  Synthetic oligonucleotide
tctttttttttttcgtt

SEQ ID NO: 4 - DNA, 17 bp, Artificial sequence
  Synthetic oligonucleotide
tcttttttgtcgttttt

SEQ ID NO: 5 - DNA, 17 bp, Artificial sequence
  Synthetic oligonucleotide
tctttttttttgtcgtt

SEQ ID NO: 6 - DNA, 22 bp, Artificial sequence
  Synthetic oligonucleotide
tcttttttttcgtttttttttt

SEQ ID NO: 7 - DNA, 22 bp, Artificial sequence
  Synthetic oligonucleotide
tctttttttttttcgttttttt

SEQ ID NO: 8 - DNA, 22 bp, Artificial sequence
  Synthetic oligonucleotide
tctttttttttttttcgttttt

SEQ ID NO: 9 - DNA, 22 bp, Artificial sequence
  Synthetic oligonucleotide
tcttttttgtcgtttttttttt

SEQ ID NO: 10 - DNA, 22 bp, Artificial sequence
  Synthetic oligonucleotide
tctttttttttgtcgttttttt

SEQ ID NO: 11 - DNA, 22 bp, Artificial sequence
  Synthetic oligonucleotide
tctttttttttttgtcgttttt

SEQ ID NO: 12 - DNA, 17 bp, Artificial sequence
  Synthetic oligonucleotide
tcttttttttcgttttt

SEQ ID NO: 13 - DNA, 22 bp, Artificial sequence
  Synthetic oligonucleotide
tcttttttttcgtttttttttt

SEQ ID NO: 14 - DNA, 22 bp, Artificial sequence
  Synthetic oligonucleotide
tcttttttcgtttttttttttt

SEQ ID NO: 15 - DNA, 20 bp, Artificial sequence
  Synthetic oligonucleotide
tccaggacttctctcaggtt

SEQ ID NO: 16 - DNA, 20 bp, Artificial sequence
  Synthetic oligonucleotide
gccaggacttctctcaggtt

SEQ ID NO: 17 - DNA, 20 bp, Artificial sequence
  Synthetic oligonucleotide
tccattacttctctcatttt

SEQ ID NO: 18 - DNA, 20 bp, Artificial sequence
  Synthetic oligonucleotide
tccaggatctctctcaggtt

SEQ ID NO: 19 - DNA, 20 bp, Artificial sequence
  Synthetic oligonucleotide
tccaggacttgtgtgaggtt

SEQ ID NO: 20 - DNA, 20 bp, Artificial sequence
  Synthetic oligonucleotide
gccaggacacctcacaggat

SEQ ID NO: 21 - DNA, 17 bp, Artificial sequence
  Synthetic oligonucleotide
tcttttttctttctttt

SEQ ID NO: 22 - DNA, 17 bp, Artificial sequence
  Synthetic oligonucleotide
tcttctttttttttttt

SEQ ID NO: 23 - DNA, 20 bp, Artificial sequence
  Synthetic oligonucleotide
tctttttcttctctcttttt

SEQ ID NO: 24 - DNA, 22 bp, Artificial sequence
  Synthetic oligonucleotide
tcttttttgtcgtttttttttt

SEQ ID NO: 25 - DNA, 22 bp, Artificial sequence
  Synthetic oligonucleotide
tcttttttgtcgtttttttttt

SEQ ID NO: 26 - DNA, 22 bp, Artificial sequence
  Synthetic oligonucleotide
tcttttttgtcgtttttttttt

SEQ ID NO: 27 - DNA, 24 bp, Artificial sequence
  Synthetic oligonucleotide
tctttttttttttttttttttttt

SEQ ID NO: 28 - DNA, 17 bp, Artificial sequence
  Synthetic oligonucleotide
tattttttttttttttt

SEQ ID NO: 29 - DNA, 17 bp, Artificial sequence
  Synthetic oligonucleotide
tgttttttttttttttt

SEQ ID NO: 30 - DNA, 17 bp, Artificial sequence
  Synthetic oligonucleotide
tnttttttttttttttt

SEQ ID NO: 31 - DNA, 17 bp, Artificial sequence
  Synthetic oligonucleotide
ncttttttttttttttt

SEQ ID NO: 32 - DNA, 17 bp, Artificial sequence
  Synthetic oligonucleotide
ncttttttttttttttt

SEQ ID NO: 33 - DNA, 17 bp, Artificial sequence
  Synthetic oligonucleotide
tnttttttttttttttt

SEQ ID NO: 34 - DNA, 17 bp, Artificial sequence
  Synthetic oligonucleotide
tcgaaaaaaaaaataaa

SEQ ID NO: 35 - DNA, 17 bp, Artificial sequence
  Synthetic oligonucleotide
tcgaaaaaaaaattaaa

SEQ ID NO: 36 - DNA, 17 bp, Artificial sequence
  Synthetic oligonucleotide
tcgaaaaaaattttaaa

SEQ ID NO: 37 - DNA, 17 bp, Artificial sequence
  Synthetic oligonucleotide
tcgaaaaattttttaaa

SEQ ID NO: 38 - DNA, 17 bp, Artificial sequence
  Synthetic oligonucleotide
tcgaaatttttttttta

SEQ ID NO: 39 - DNA, 17 bp, Artificial sequence
  Synthetic oligonucleotide
tcgtaaaaaaaaaaaaa

SEQ ID NO: 40 - DNA, 17 bp, Artificial sequence
  Synthetic oligonucleotide
tcgtttaaaaaaaaaaa

SEQ ID NO: 41 - DNA, 17 bp, Artificial sequence
  Synthetic oligonucleotide
tcgaaaaaaaaaaaaaa

SEQ ID NO: 42 - DNA, 17 bp, Artificial sequence
  Synthetic oligonucleotide
tcgtttttttttttttt

SEQ ID NO: 43 - DNA, 17 bp, Artificial sequence
  Synthetic oligonucleotide
ttttttttttttttttt

SEQ ID NO: 44 - DNA, 17 bp, Artificial sequence
  Synthetic oligonucleotide
tttttttttttttttcg

SEQ ID NO: 45 - DNA, 17 bp, Artificial sequence
  Synthetic oligonucleotide
tttcgtttttttttttt

SEQ ID NO: 46 - DNA, 17 bp, Artificial sequence
  Synthetic oligonucleotide
ttttttcgttttttttt

SEQ ID NO: 47 - DNA, 17 bp, Artificial sequence
  Synthetic oligonucleotide
tttttttttcgtttttt

SEQ ID NO: 48 - DNA, 17 bp, Artificial sequence
  Synthetic oligonucleotide
ttcgttttttttttttt

SEQ ID NO: 49 - DNA, 17 bp, Artificial sequence
  Synthetic oligonucleotide
ttttcgttttttttttt

SEQ ID NO: 50 - DNA, 17 bp, Artificial sequence
  Synthetic oligonucleotide
tttttcgtttttttttt

SEQ ID NO: 51 - DNA, 23 bp, Artificial sequence
  Synthetic oligonucleotide
tttttttttcgtttttttttttt

SEQ ID NO: 52 - DNA, 17 bp, Artificial sequence
  Synthetic oligonucleotide
tttttttttngtttttt

SEQ ID NO: 53 - DNA, 17 bp, Artificial sequence
  Synthetic oligonucleotide
attttttttcgtttttt

SEQ ID NO: 54 - DNA, 24 bp, Artificial sequence
  Synthetic oligonucleotide
tcgtcgttttgtcgttttgtcgtt

SEQ ID NO: 55 - DNA, 17 bp, Artificial sequence
  Synthetic oligonucleotide
tcgcccccccccccccc

SEQ ID NO: 56 - DNA, 17 bp, Artificial sequence
  Synthetic oligonucleotide
acgtttttttttttttt

SEQ ID NO: 57 - DNA, 17 bp, Artificial sequence
  Synthetic oligonucleotide
ccgtttttttttttttt

SEQ ID NO: 58 - DNA, 17 bp, Artificial sequence
  Synthetic oligonucleotide
gcgtttttttttttttt

SEQ ID NO: 59 - DNA, 17 bp, Artificial sequence
  Synthetic oligonucleotide
ttgtttttttttttttt

SEQ ID NO: 60 - DNA, 22 bp, Artificial sequence
  Synthetic oligonucleotide
tcgtcgttttcggcgcgcgccg

SEQ ID NO: 61 - DNA, 20 bp, Artificial sequence
  Synthetic oligonucleotide
tccatgacgttcctgacgtt

SEQ ID NO: 62 - DNA, 24 bp, Artificial sequence
  Synthetic oligonucleotide
ttgcgtgcgttttgacgttttttt

SEQ ID NO: 63 - DNA, 18 bp, Artificial sequence
  Synthetic oligonucleotide
tcacatgtggagccgcgt

SEQ ID NO: 64 - DNA, 20 bp, Artificial sequence
  Synthetic oligonucleotide
tccaagacgttcctgatgct

SEQ ID NO: 65 - DNA, 20 bp, Artificial sequence
  Synthetic oligonucleotide
tccataacgttcctgatgct

SEQ ID NO: 66 - DNA, 20 bp, Artificial sequence
  Synthetic oligonucleotide
tccataacgttcctgatgct

SEQ ID NO: 67 - DNA, 20 bp, Artificial sequence
  Synthetic oligonucleotide
tccatattgcacctgatgct

SEQ ID NO: 68 - DNA, 20 bp, Artificial sequence
  Synthetic oligonucleotide
tccatcacgtgcctgatgct

SEQ ID NO: 69 - DNA, 20 bp, Artificial sequence
  Synthetic oligonucleotide
tccatcacgtgcctgatgct

SEQ ID NO: 70 - DNA, 45 bp, Artificial sequence
  Synthetic oligonucleotide
tccatcgccaaggagatcgagctggaggatccgtacgagaagatc

SEQ ID NO: 71 - DNA, 20 bp, Artificial sequence
  Synthetic oligonucleotide
tccatgacggtcctgatgct

SEQ ID NO: 72 - DNA, 20 bp, Artificial sequence
  Synthetic oligonucleotide
tccatgacggtcctgatgct

SEQ ID NO: 73 - DNA, 20 bp, Artificial sequence
  Synthetic oligonucleotide
tccatgacgtccctgatgct

SEQ ID NO: 74 - DNA, 20 bp, Artificial sequence
  Synthetic oligonucleotide
tccatgacgtccctgatgct

SEQ ID NO: 75 - DNA, 20 bp, Artificial sequence
  Synthetic oligonucleotide
tccatgacgttcctgatgct

SEQ ID NO: 76 - DNA, 20 bp, Artificial sequence
  Synthetic oligonucleotide
tccatgacgttcctgatgct

SEQ ID NO: 77 - DNA, 20 bp, Artificial sequence
  Synthetic oligonucleotide
tccatgacgttcctgatgct

SEQ ID NO: 78 - DNA, 20 bp, Artificial sequence
  Synthetic oligonucleotide
tccatgacgttcctgatgct

SEQ ID NO: 79 - DNA, 20 bp, Artificial sequence
  Synthetic oligonucleotide
tccatgacgttcctgatgct

SEQ ID NO: 80 - DNA, 20 bp, Artificial sequence
  Synthetic oligonucleotide
tccatgacgttcctgatgct

SEQ ID NO: 81 - DNA, 20 bp, Artificial sequence
  Synthetic oligonucleotide
tccatgacgttcctgatgct

SEQ ID NO: 82 - DNA, 20 bp, Artificial sequence
  Synthetic oligonucleotide
tccatgagcttcctgagtct

SEQ ID NO: 83 - DNA, 20 bp, Artificial sequence
  Synthetic oligonucleotide
tccatgagcttcctgatgct

SEQ ID NO: 84 - DNA, 20 bp, Artificial sequence
  Synthetic oligonucleotide
tccatgagcttcctgatgct

SEQ ID NO: 85 - DNA, 20 bp, Artificial sequence
  Synthetic oligonucleotide
tccatgccggtcctgatgct

SEQ ID NO: 86 - DNA, 20 bp, Artificial sequence
  Synthetic oligonucleotide
tccatgccggtcctgatgct

SEQ ID NO: 87 - DNA, 20 bp, Artificial sequence
  Synthetic oligonucleotide
tccatgctggtcctgatgct

SEQ ID NO: 88 - DNA, 20 bp, Artificial sequence
  Synthetic oligonucleotide
tccatgctggtcctgatgct

SEQ ID NO: 89 - DNA, 20 bp, Artificial sequence
  Synthetic oligonucleotide
tccatggcggtcctgatgct

SEQ ID NO: 90 - DNA, 20 bp, Artificial sequence
  Synthetic oligonucleotide
tccatggcggtcctgatgct

SEQ ID NO: 91 - DNA, 20 bp, Artificial sequence
  Synthetic oligonucleotide
tccatgtcgatcctgatgct

SEQ ID NO: 92 - DNA, 20 bp, Artificial sequence
  Synthetic oligonucleotide
tccatgtcgatcctgatgct

SEQ ID NO: 93 - DNA, 20 bp, Artificial sequence
  Synthetic oligonucleotide
tccatgtcgctcctgatgct

SEQ ID NO: 94 - DNA, 20 bp, Artificial sequence
  Synthetic oligonucleotide
tccatgtcgctcctgatgct

SEQ ID NO: 95 - DNA, 20 bp, Artificial sequence
  Synthetic oligonucleotide
tccatgtcggtcctgatgct

SEQ ID NO: 96 - DNA, 20 bp, Artificial sequence
  Synthetic oligonucleotide
tccatgtcggtcctgatgct

SEQ ID NO: 97 - DNA, 20 bp, Artificial sequence
  Synthetic oligonucleotide
tccatgtcggtcctgatgct

SEQ ID NO: 98 - DNA, 20 bp, Artificial sequence
  Synthetic oligonucleotide
tccatgtcggtcctgatgct

SEQ ID NO: 99 - DNA, 20 bp, Artificial sequence
  Synthetic oligonucleotide
tccatgtcggtcctgctgat

SEQ ID NO: 100 - DNA, 20 bp, Artificial sequence
  Synthetic oligonucleotide
tccatgtcggtnctgatgct

SEQ ID NO: 101 - DNA, 20 bp, Artificial sequence
  Synthetic oligonucleotide
tccatgtcgttcctgatgct

SEQ ID NO: 102 - DNA, 20 bp, Artificial sequence
  Synthetic oligonucleotide
tccatgtcgttcctgatgct

SEQ ID NO: 103 - DNA, 20 bp, Artificial sequence
  Synthetic oligonucleotide
tccatgtcgttcctgatgct

SEQ ID NO: 104 - DNA, 20 bp, Artificial sequence
  Synthetic oligonucleotide
tccatgtnggtcctgatgct

SEQ ID NO: 105 - DNA, 20 bp, Artificial sequence
  Synthetic oligonucleotide
tccatgtngttcctgatgct

SEQ ID NO: 106 - DNA, 21 bp, Artificial sequence
  Synthetic oligonucleotide
tcccccatgccgccctccggg

SEQ ID NO: 107 - DNA, 30 bp, Artificial sequence
  Synthetic oligonucleotide
tccgctgacgtcgccgcccagatggcctcc

SEQ ID NO: 108 - DNA, 15 bp, Artificial sequence
  Synthetic oligonucleotide
tcctcctcctcctcc

SEQ ID NO: 109 - DNA, 18 bp, Artificial sequence
  Synthetic oligonucleotide
tcggcggtgaagaagact

SEQ ID NO: 110 - DNA, 20 bp, Artificial sequence
  Synthetic oligonucleotide
tcggtcaacgttgagatgct

SEQ ID NO: 111 - DNA, 30 bp, Artificial sequence
  Synthetic oligonucleotide
tcggtgaacgttatgtcgcaggacccggtc

SEQ ID NO: 112 - DNA, 30 bp, Artificial sequence
  Synthetic oligonucleotide
tcggtgaccggtatgtcgcaggacccggtc

SEQ ID NO: 113 - DNA, 30 bp, Artificial sequence
  Synthetic oligonucleotide
tcggtgagcgctatgtcgcaggacccggtc

SEQ ID NO: 114 - DNA, 30 bp, Artificial sequence
  Synthetic oligonucleotide
tcggtgcagggaatgtcgcaggacccggtc

SEQ ID NO: 115 - DNA, 45 bp, Artificial sequence
  Synthetic oligonucleotide
tcggtgcagggaatgtcgcaggacccggtcgcggtggcggcctcg

SEQ ID NO: 116 - DNA, 30 bp, Artificial sequence
  Synthetic oligonucleotide
tcggtgcagggaatgtcgcaggacgacgtc

SEQ ID NO: 117 - DNA, 30 bp, Artificial sequence
  Synthetic oligonucleotide
tcggtggacgtcatgtcgcaggacccggtc

SEQ ID NO: 118 - DNA, 30 bp, Artificial sequence; identical to SEQ ID NO: 117
  Synthetic oligonucleotide

SEQ ID NO: 119 - DNA, 30 bp, Artificial sequence
  Synthetic oligonucleotide
tcggtggactgcatgtcgcaggacccggtc

SEQ ID NO: 120 - DNA, 30 bp, Artificial sequence; identical to SEQ ID NO: 119
  Synthetic oligonucleotide

SEQ ID NO: 121 - DNA, 15 bp, Artificial sequence
  Synthetic oligonucleotide
tcgtcgctgtctccg

SEQ ID NO: 122 - DNA, 21 bp, Artificial sequence
  Synthetic oligonucleotide
tcgtcgctgtctccgcttctt

SEQ ID NO: 123 - DNA, 27 bp, Artificial sequence
  Synthetic oligonucleotide
tcgtcgctgtctccgcttcttcttgcc

SEQ ID NO: 124 - DNA, 27 bp, Artificial sequence
  Synthetic oligonucleotide
tcgtcgctgtctccgcttcttcttgcc

SEQ ID NO: 125 - DNA, 27 bp, Artificial sequence
  Synthetic oligonucleotide
tcgtcgctgtctccgcttcttcttgcc

SEQ ID NO: 126 - DNA, 28 bp, Artificial sequence
  Synthetic oligonucleotide
tcgtcgctgtctccgcttcttcttgcca

SEQ ID NO: 127 - DNA, 16 bp, Artificial sequence
  Synthetic oligonucleotide
tcgtcggggggggggg

SEQ ID NO: 128 - DNA, 12 bp, Artificial sequence
  Synthetic oligonucleotide
tcgtcgtcgtcg

SEQ ID NO: 129 - DNA, 15 bp, Artificial sequence
  Synthetic oligonucleotide
tcgtcgtcgtcgtcg

SEQ ID NO: 130 - DNA, 20 bp, Artificial sequence
  Synthetic oligonucleotide
tctccatgatggttttatcg

SEQ ID NO: 131 - DNA, 18 bp, Artificial sequence
  Synthetic oligonucleotide
tctcccagcgtgcgccat

SEQ ID NO: 132 - DNA, 18 bp, Artificial sequence
  Synthetic oligonucleotide
tctcccagcgtgcgccat

SEQ ID NO: 133 - DNA, 18 bp, Artificial sequence
  Synthetic oligonucleotide
tctcccagngtgngccat

SEQ ID NO: 134 - DNA, 18 bp, Artificial sequence
  Synthetic oligonucleotide
tcttctgccccctgtgca

SEQ ID NO: 135 - DNA, 21 bp, Artificial sequence
  Synthetic oligonucleotide
tgacgtttgacgtttgacgtt

SEQ ID NO: 136 - DNA, 22 bp, Artificial sequence
  Synthetic oligonucleotide
tgactgtgaacgttcgagatga

SEQ ID NO: 137 - DNA, 18 bp, Artificial sequence
  Synthetic oligonucleotide
tgatcttccatctattag

SEQ ID NO: 138 - DNA, 18 bp, Artificial sequence
  Synthetic oligonucleotide
tgcacagggggcagaaga

SEQ ID NO: 139 - DNA, 15 bp, Artificial sequence
  Synthetic oligonucleotide
tggtggtggtggtgg

SEQ ID NO: 140 - DNA, 20 bp, Artificial sequence
  Synthetic oligonucleotide
ttgcttccatcttcctcgtc

SEQ ID NO: 141 - DNA, 26 bp, Artificial sequence
  Synthetic oligonucleotide
ttggtgaagctaacgttgaggggcat

SEQ ID NO: 142 - DNA, 24 bp, Artificial sequence
  Synthetic oligonucleotide
tcgtcgttttgtcgtttttttcga

SEQ ID NO: 143 - DNA, 24 bp, Artificial sequence
  Synthetic oligonucleotide
tcgtcgtttcgtcgttttgtcgtt

(19) United States
(12) Patent Application Publication
(10) Pub. No.: US 2006/0019916 A1
(43) Pub. Date: Jan. 26, 2006

(54) Immunostimulatory nucleic acids for inducing IL-10 responses

(75) Inventors: Arthur M. Krieg, Wellesley, MA (US); Jorg Vollmer, Dusseldorf (DE)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Coley Pharmaceutical Group, Inc., Wellesley, MA (US)

(21) Appl. No.: 11/099,683

(22) Filed: Apr. 4, 2005

Related U.S. Application Data
(60) Provisional application No. 60/558,951, filed on Apr. 2, 2004.

(51) Int. Cl. A61K 48/00; C07H 21/04

(52) U.S. Cl. 514/44; 536/23.1

(57) ABSTRACT

The invention relates to methods and products for inducing IL-10 expression using immunostimulatory nucleic acids. In particular, the invention relates to methods and products for inducing IL-10 expression without inducing high levels of IFN-α expression. IL-10-inducing immunostimulatory nucleic acids preferably include a TC dinucleotide at the 5′ end and a CG dinucleotide towards the 3′ end, but not near the 5′ end. The invention is useful for treating and preventing disorders associated with a Th1 or Th2 immune response or for promoting a T regulatory cell environment suitable for suppressing inappropriate immune responses (e.g., for controlling or suppressing excessive immune responses).

### RELATED APPLICATIONS

This application claims priority to U.S. Provisional Patent Application filed Apr. 2, 2004, entitled “IMMUNOSTIMULATORY NUCLEIC ACIDS FOR INDUCING IL-10 RESPONSES”, Ser. No. 60/558,951, the contents of which are incorporated by reference herein in their entirety.

### FIELD OF THE INVENTION

The present invention relates generally to immunostimulatory nucleic acids, and particularly to CpG containing immunostimulatory nucleic acids and their therapeutic uses.

### BACKGROUND OF INVENTION

The existence of functionally polarized T cell responses based on the profile of cytokines secreted by CD4+ T helper (Th) cells has been well established. In general, Th1 cells secrete interferon-gamma (IFN-γ), interleukin (IL)-2, and tumor necrosis factor-beta (TNFβ), and are important in macrophage activation, the generation of both humoral and cell-mediated immune responses and phagocyte-dependent protective responses. Th2 cells secrete IL-4, IL-5, IL-10, and IL-13 and are more important in the generation of humoral immunity, eosinophil activation, regulation of cell-mediated immune responses, control of macrophage function and the stimulation of particular Ig isotypes (Morel et al., 1998, Romagnani, 1999). Th1 cells generally develop following infections by intracellular pathogens, whereas Th2 cells predominate in response to intestinal nematodes. In addition to their roles in protective immunity, Th1 and Th2 cells are responsible for different types of immunopathological disorders. For example, Th1 cells tend to predominate in organ-specific autoimmune disorders, Crohn's disease, Helicobacter pylori-induced peptic ulcer, acute solid organ allograft rejection, and unexplained recurrent abortion, whereas Th2 cells tend to predominate in Omenn's syndrome, systemic lupus erythematosus, transplantation tolerance, chronic graft versus host disease, idiopathic pulmonary fibrosis, and progressive systemic sclerosis, and are involved in triggering of allergic reactions including most asthma (Romagnani 1999, Singh et al., 1999). In many diseases, such as lupus, there is evidence for both a Th1 and Th2 component contributing to pathogenesis either at the same or different times during disease development.

An additional type of T cell response was observed when T cells were activated in the presence of interleukin 10 (IL-10). IL-10 activation results in the generation of a T cell subset known as regulatory T cells. Regulatory T cells have a cytokine profile that differs from both the Th1 and Th2 cytokine profiles. Regulatory T cells were also observed to have inhibitory effects on Ag-specific or Ag-nonspecific T cell activation, including both Th1 and Th2 responses.

In recent years, a number of studies have demonstrated the ability of unmethylated CpG dinucleotides (i.e., the cytosine is unmethylated) within the context of certain flanking sequences (CpG motifs) to stimulate both innate and specific immune responses. Such sequences are commonly found in bacterial DNA which is immunostimulatory. Similar immunostimulation is also possible with synthetic oligodeoxynucleotides (ODN) containing CpG motifs (CpG ODN). It has been demonstrated that CpG DNA can induce stimulation of B cells to proliferate and secrete immunoglobulin (Ig), IL-6 and IL-12, and to be protected from apoptosis (Krieg et al., 1995, Yi et al., 1996, Klinman et al., 1996). These effects contribute to the ability of CpG DNA to have adjuvant activity. In addition, CpG DNA enhances expression of class II MHC and B7 co-stimulatory molecules (Davis et al., 1998, Sparwasser et al., 1998), that leads to improved antigen presentation. Furthermore, CpG DNA also directly activates dendritic cells in mice to secrete various cytokines and chemokines (Uhlmann and Vollmer, 2003) that can provide T-helper functions. These in vitro effects were believed to be specific to the unmethylated CpG motifs since they were not induced by methylated bacterial DNA or in general by ODN that do not contain unmethylated CpG motifs.

Immunization of animals against a variety of antigens delivered both parenterally and mucosally demonstrate that addition of CpG ODN induces more Th1-dominated responses as indicated by strong cytotoxic T lymphocytes (CTL) stimulation, high levels of IgG2a antibodies, and predominantly Th1 cytokines (e.g., IL-12 and IFN-γ but not IL-4 or IL-5) (Klinman et al., 1996, Davis et al., 1998, Roman et al., 1997, Chu et al., 1997, Lipford et al., 1997, Weiner et al., 1997, McCluskie and Davis, 1998, 1999).

In contrast, immunization experiments using nucleic acids lacking a CpG demonstrate that mucosal administration of these nucleic acids can induce a Th2-dominated response.

### SUMMARY OF THE INVENTION

The invention provides a subset of CpG containing nucleic acids that induce high levels of interleukin 10 (IL-10) expression without significant induction of interferon alpha (IFN-α) expression and type I interferon-mediated effects.

In one aspect, the invention provides CpG containing immunostimulatory nucleic acids that include a 5′ TC dinucleotide separated from one or more CpG dinucleotides located towards the 3′ end of the nucleic acid. In preferred embodiments, the nucleic acid contains only one CpG dinucleotide.

In one aspect, the CpG immunostimulatory nucleic acids of the invention are useful for stimulating IL-10 expression without stimulating IFN-α expression and type I interferon-mediated effects.

In another aspect, the CpG immunostimulatory nucleic acids of the invention are useful for obtaining a regulatory T cell response. In particular, the CpG immunostimulatory nucleic acids are useful for treating diseases or conditions where a regulatory T cell response is favorable.

In another aspect, the CpG immunostimulatory nucleic acids of the invention are useful for obtaining a regulatory B cell response. In particular, the CpG immunostimulatory nucleic acids are useful for treating diseases or conditions where a regulatory B cell response is favorable.

In another aspect, the CpG immunostimulatory nucleic acids of the invention are useful for stimulating B cells. In particular, the CpG immunostimulatory nucleic acids are useful for treating diseases or conditions where B cell stimulation is favorable.

In another aspect, the CpG immunostimulatory nucleic acids of the invention are useful for obtaining a regulatory B cell response. In particular, the CpG immunostimulatory nucleic acids are useful for treating diseases or conditions where a regulatory B cell response is favorable.

In another aspect, the CpG immunostimulatory nucleic acids of the invention are useful to reduce or minimize a host subject's rejection of an organ transplant or tissue graft.

In another aspect, the CpG immunostimulatory nucleic acids of the invention are useful to treat asthma, allergy, autoimmune diseases, and other inflammatory disorders.

In another aspect, the CpG immunostimulatory nucleic acids of the invention are useful for antigen-specific vaccinations in patients with an autoimmune disease.

In another aspect, the invention is an oligonucleotide chosen from: a) 5′ XYN₁YZN₂ 3′, wherein 5′ designates the 5′ end of the oligonucleotide and 3′ designates the 3′ end of the oligonucleotide, wherein X is a T or modified T nucleotide, wherein Y is a C or modified C nucleotide, wherein Z is a G or modified G nucleotide, wherein N₁ and N₂ are polynucleotides that do not include a CG dinucleotide, wherein N₁ does not include 5′ Z nucleotide, and wherein a 3′ polynucleotide consisting of the YZ dinucleotide and the N₂ polynucleotide contains a number of nucleotides that is at most 45% of the number of nucleotides in the oligonucleotide; and b) 5′ XY N₁YZ N₂ 3′, wherein 5′ designates the 5′ end of the oligonucleotide and 3′ designates the 3′ end of the oligonucleotide, wherein X is a T or modified T nucleotide, wherein Y is a C or modified C nucleotide, wherein Z is a G or modified G nucleotide, wherein N₁ is a polynucleotide of 5 to 10 nucleotides, wherein N₁ does not include a CG dinucleotide, wherein N₁ does not include 5′ Z nucleotide, and wherein N₂ is a polynucleotide of 5 to 30 nucleotides.

In some embodiments, the oligonucleotide includes at least 1 modified internucleotide linkage. In other embodiments, the oligonucleotide includes at least 50% modified internucleotide linkages. In other embodiments, all internucleotide linkages of the oligonucleotide are modified. In yet other embodiments, between 0% and 10%, between 10% and 20%, between 20% and 30%, between 30% and 40%, between 40% and 50%, between 50% and 60%, between 60% and 70%, between 70% and 80%, between 80% and 90%, or between 90% and 100% modified internucleotide linkages. In other embodiments, the oligonucleotide consists of 10 to 100 nucleotides. In some embodiments, the modified internucleotide linkage is a phosphorothioate linkage. In some embodiments, the oligonucleotide comprises a phosphodiester linkage between a 5′ C. nucleotide and a 3′ G nucleotide. In other embodiments, the oligonucleotide comprises a R-phosphorothioate linkage between a 5° C. nucleotide and a 3′ G nucleotide.

In some embodiments, Y is a modified C nucleotide comprising a modified cytosine base selected from the group consisting of 5-substituted cytosines, 6-substituted cytosines, N4-substituted cytosines, cytosine analogs with condensed ring systems, uracil, uracil derivatives, a universal base, an aromatic ring system, and a hydrogen atom. In other embodiments, Y is a modified C nucleotide comprising a modified cytosine base selected from the group consisting of 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, unsubstituted or substituted 5-alkynyl-cytosine, N4-ethyl-cytosine, 5-aza-cytosine, 2-mercapto-cytosine, isocytosine, pseudo-isocytosine, N,N′-propylene cytosine or phenoxazine, 5-fluoro-uracil, 5-bromo-uracil, 5-bromovinyl-uracil, 4-thio-uracil, 5-hydroxy-uracil, 5-propynyl-uracil, 3-nitropyrrole, P-base, fluorobenzene, and difluorobenzene.

In some embodiments, Z is a modified G nucleotide comprising a modified guanine base selected from the group consisting of 7-deazaguanine, 7-deaza-7-substituted guanine, 7-deaza-7-(C2-C6)alkynylguanine, 7-deaza-8-substituted guanine, hypoxanthine, N2-substituted guanines, N2-methyl-guanine, 5-amino-3-methyl-3H,6H-thiazolo[4,5-d]pyrimidine-2,7-dione, 2,6-diaminopurine, 2-aminopurine, purine, indole, inosine, adenine, substituted adenines, N6-methyl-adenine, 8-oxo-adenine, 8-substituted guanine, 8-hydroxyguanine, 8-bromoguanine, 6-thioguanine, a universal base, 4-methyl-indole, 5-nitro-indole, K-base, an aromatic ring system, benzimidazole, dichloro-benzimidazole, 1-methyl-1H-[1,2,4]triazole-3-carboxylic acid amide, and a hydrogen atom.

In some embodiments, the oligonucleotide comprises a 3′-3′ linkage with one or two accessible 5′ ends.

In some embodiments, the oligonucleotide comprises a nucleotide sequence that does not contain an optimal CpG hexameric sequence. In other embodiments, the oligonucleotide comprises a nucleotide sequence that does not contain a palindromic sequence. In other embodiments, the oligonucleotide does not form a stable secondary structure.

In some embodiments, the oligonucleotide is conjugated to a moiety selected from the group consisting of antigens and cytokines. In some embodiments, the antigen can be selected from the group consisting of infectious disease antigens. In some embodiments, the cytokine can be selected from the group consisting of IL-4, IL-10, IL-12.

In one embodiment, the oligonucleotide has the following structure: 5′ T*C*T*T*T*T*T*T*G*T*C*G*T*T*T*T*T 3′ (SEQ ID NO:4) and wherein * refers to a phosphorothioate linkage. In another embodiment, the oligonucleotide has the following structure: 5′ T*T*G*C*G*T*G*C*G*T*T*T*T*G*A*C*G*T*T*T*T*T*T*T 3′(SEQ ID NO:62) and wherein * refers to a phosphorothioate linkage. In another embodiment, the oligonucleotide has the following structure: 5′ T*C*T*T*T*T*T*T*T*T*C*G*T*T*T*T*T 3′ (SEQ ID NO:2) and wherein * refers to a phosphorothioate linkage.

In some embodiments, N₁ is a poly-T polynucleotide. In other embodiments, N₂ is a poly-T polynucleotide. Both N₁ and N₂ can also be poly-T polynucleotides. The poly-T polynucleotide can contain one or more modified T nucleotides. In preferred embodiments, the poly-T polynucleotide contains between 5 and 20 T nucleotides, between 5 and 10 T nucleotides, more than 20 T nucleotides, or at least 55% T nucleotides.

In another aspect, the invention is a pharmaceutical composition including an oligonucleotide described herein in combination with a therapeutic agent selected from the group consisting of chemotherapeutic agents, radiotherapeutic agents, monoclonal antibodies, and anticancer agents. In some embodiments, the pharmaceutical composition comprises an oligonucleotide in combination with a polycation carrier.

In another aspect, the invention is a method of specifically increasing IL-10 expression relative to IFN-α expression in a subject, including the step of administering an oligonucleotide or a pharmaceutical composition of the invention to a subject in whom inducing a T regulatory response may be beneficial. In preferred embodiments, the step of administering is selected from the group consisting of respiratory, oral, topical, subcutaneous, and intra-venous administrations.

In another aspect, the invention is a method of inducing an antigen-specific regulatory T or B cell response in a subject, including the step of: administering an immunostimulatory nucleic acid or composition of the invention to a subject exposed to an antigen. In some embodiments, the antigen is administered to the subject along with the immunostimulatory nucleic acid or composition. In other embodiments, the antigen is administered to the subject after the immunostimulatory nucleic acid or composition. In other embodiments, the antigen is present in a food and the subject is exposed to the antigen by ingesting the food. In yet other embodiments, the antigen is inhaled by the subject.

In another aspect, the invention is a method of treating an allergy or asthma, including the steps of exposing a subject to an allergen and administering an immunostimulatory nucleic acid or composition of the invention to the subject, wherein the immunostimulatory nucleic acid or composition is administered in an amount sufficient to prevent or alleviate an allergic response to the allergen in the subject. In some embodiments, the method also includes administering IL-10 to the subject. In some embodiments, the subject has or is at risk of developing allergic asthma.

In another aspect, the invention is a method of treating an autoimmune disease in a subject, including the steps of exposing a subject to a self antigen and administering an immunostimulatory nucleic acid or composition of the invention to the subject, wherein the immunostimulatory nucleic acid or composition is administered in an amount sufficient to prevent or treat an autoimmune disease in the subject. In some embodiments, the method also includes administering IL-10 to the subject. In some embodiments, the autoimmune disease is arthritis, multiple sclerosis, Type 1 diabetes mellitus, Multiple sclerosis, Myasthenia gravis, Autoimmune neuropathies, such as Guillain-Barre, Autoimmune uveitis, Autoimmune hemolytic anemia, Pernicious anemia, Autoimmune thrombocytopenia, Temporal arteritis, Anti-phospholipid syndrome, Psoriasis, Pemphigus vulgaris, Vasculitides such as Wegener's granulomatosis, Vitiligo, Crohn's Disease, Ulcerative colitis, Primary biliary cirrhosis, Autoimmune hepatitis, Type 1 or immune-mediated, diabetes mellitus, Grave's Disease, Hashimoto's thyroiditis, Autoimmune oophoritis and orchitis, Autoimmune disease of the adrenal gland, Rheumatoid arthritis, Systemic lupus erythematosus, Scleroderma, Polymyositis, dermatomyositis, Spondyloarthropathies, such as ankylosing spondylitis, or Sjogren's syndrome. In some embodiments, the autoimmune disease is caused by an infection, for example Lyme disease.

In another aspect, the invention is a method of reducing an antigen-specific response to an implant in a subject, including the steps of exposing a subject to an implant antigen and administering an immunostimulatory nucleic acid or composition of the invention to the subject, wherein the immunostimulatory nucleic acid or composition is administered in an amount sufficient to prevent or reduce an antigen-specific response to the implant in the subject. In some embodiments, the method also includes administering IL-10 to the subject. In some embodiments, the implant is an autologous tissue implant. In other embodiments, the implant is a non-autologous tissue implant. In other embodiments, the implant is a recombinant cellular implant. In other embodiments, the implant is a synthetic implant.

In some embodiments, the invention does not include one or more nucleic acids, or use thereof, having one or more of the following sequences (shown 5′ to 3′):

| | |
|---|---|
| TCAAGGCT; TCAAGGTTT; TGAACGTT; | (SEQ ID NO:63) |
| TCAAGCTT; TCAAGCTT; TCACATGTGG | |
| AGCCGCGT; | |
| TCACGGTT; TCAGCGCT; TCAGCGCT; | (SEQ ID NO:64) |
| TGATGGAT; TCATCGAT; TCCAAGACGTTCC | |
| TGATGCT; | |
| TCCATAACGTTCCTGATGGT; | (SEQ ID NO:65) |
| TCCATAACGTTCCTGATGCT; | (SEQ ID NO:66) |
| TCCATATTGCACCTGATGCT | (SEQ ID NO:67) |
| TCCATCACGTGCCTGATGCT; | (SEQ ID NO:68) |
| TGCATCACGTGCCTGATGCT; | (SEQ ID NO:69) |
| TCCATCGCCAAGGAGATCGAGCTGGAGGATCCG | (SEQ ID NO:70) |
| TACGAGAAGATC; | |
| TCCATGACGGTCGTGATGCT; | (SEQ ID NO:71) |
| TCCATGACGGTGCTGATGCT; | (SEQ ID NO:72) |
| TCCATGACGTCCCTGATGGT; | (SEQ ID NO:73) |
| TCCATGACGTCCCTGATGCT; | (SEQ ID NO:74) |
| TCCATGAGGTTGCTGATGCT; | (SEQ ID NO:75) |
| TCCATGAGGTTCCTGATGGT; | (SEQ ID NO:76) |
| TCCATGACGTTCCTGATGCT; | (SEQ ID NO:77) |
| TCCATGACGTTCCTGATGCT; | (SEQ ID NO:78) |
| TCCATGACGTTCCTGATGGT; | (SEQ ID NO:79) |
| TCCATGACGTTCCTGATGCT; | (SEQ ID NO:80) |
| TCCATGACGTTCCTGATGCT; | (SEQ ID NO:81) |
| TCCATGAGGTTCCTGAGTGT; | (SEQ ID NO:82) |
| TCCATGAGCTTCCTGATGCT; | (SEQ ID NO:83) |
| TCCATGAGCTTCCTGATGCT; | (SEQ ID NO:84) |
| TCCATGCCGGTCCTGATGCT; | (SEQ ID NO:85) |
| TCCATGCCGGTCGTGATGCT; | (SEQ ID NO:86) |
| TCCATGCTGGTCCTGATGGT; | (SEQ ID NO:87) |
| TCCATGCTGGTCCTGATGCT; | (SEQ ID NO:88) |
| TCCATGGCGGTCCTGATGGT; | (SEQ ID NO:89) |
| TCCATGGCGGTCCTGATGCT; | (SEQ ID NO:90) |
| TCCATGTCGATCCTGATGCT; | (SEQ ID NO:91) |
| TCCATGTCGATCCTGATGGT; | (SEQ ID NO:92) |
| TCCATGTCGCTGCTGATGCT; | (SEQ ID NO:93) |
| TCCATGTCGCTCCTGATGCT; | (SEQ ID NO:94) |
| TCCATGTCGGTCCTGATGGT; | (SEQ ID NO:95) |
| TCCATGTCGGTCCTGATGCT; | (SEQ ID NO:96) |
| TCCATGTGGGTCCTGATGCT; | (SEQ ID NO:97) |
| TCCATGTCGGTCCTGATGGT; | (SEQ ID NO:98) |
| TCCATGTCGGTCCTGCTGAT; | (SEQ ID NO:99) |
| TCCATGTCGGTZCTGATGCT; | (SEQ ID NO:100) |
| TCCATGTGGTTCCTGATGCT; | (SEQ ID NO:101) |
| TGCATGTGGTTCCTGATGCT; | (SEQ ID NO:102) |
| TCCATGTCGTTCCTGATGGT; | (SEQ ID NO:103) |
| TCCATGTZGGTCCTGATGGT; | (SEQ ID NO:104) |
| TCCATGTZGTTCCTGATGCT; | (SEQ ID NO:105) |
| TCCCCCATGCCGCCCTCCGGG; | (SEQ ID NO:106) |
| TCCGCGTT; TCCGCTGAGGTCGCCGCCCAGATG | (SEQ ID NO:107) |
| GCCTCC; | |
| TCGTCCTCGTCCTCC; | (SEQ ID NO:108) |
| TCGAGGTG; TCGGCGGTGAAGAAGACT; | (SEQ ID NO:109) |
| TCGGTCAACGTTGAGATGCT; | (SEQ ID NO:110) |
| TCGGTGAACGTTATGTCGCAGGACCCGGTC; | (SEQ ID NO:111) |
| TCGGTGACCGGTATGTCGCAGGACCCGGTC; | (SEQ ID NO:112) |
| TCGGTGAGCGCTATGTCGCAGGACCGGGTC; | (SEQ ID NO:113) |
| TCGGTGCAGGGAATGTCGCAGGACCCGGTC; | (SEQ ID NO:114) |
| TCGGTGCAGGGAATGTCGCAGGACCCGGTCGCGG | (SEQ ID NO:115) |
| TGGCGGCCTCG; | |
| TGGGTGCAGGGAATGTCGCAGGACGAGGTC; | (SEQ ID NO:116) |
| TCGGTGGACGTCATGTCGCAGGACCCGGTC; | (SEQ ID NO:117) |
| TCGGTGGACGTCATGTCGCAGGACCCGGTC; | (SEQ ID NO:118) |
| TCGGTGGACTGGATGTCGCAGGACCCGGTC; | (SEQ ID NO:119) |
| TCGGTGGACTGCATGTCGGAGGACCCGGTC; | (SEQ ID NO:120) |
| TCGTCG; TCGTCGCTGTCTCCG; | (SEQ ID NO:121) |
| TCGTCGCTGTCTCCGCTTCTT; | (SEQ ID NO:122) |
| TCGTCGCTGTCTCCGCTTCTTCTTGCC; | (SEQ ID NO:123) |
| TCGTCGCTGTCTCCGGTTCTTCTTGCC; | (SEQ ID NO:124) |
| TCGTCGGTGTCTCCGCTTCTTCTTGCC; | (SEQ ID NO:125) |
| TCGTCGCTGTCTCCGCTTGTTCTTGGGA; | (SEQ ID NO:126) |
| TCGTCGGGGGGGGGGG; | (SEQ ID NO:127) |
| TCGTCGTCG; TCGTCGTCGTCG | (SEQ ID NO:128) |
| TCGTCGTCGTCGTCG; | (SEQ ID NO:129) |
| TCTCCATGATGGTTTTATGG; | (SEQ ID NO:130) |
| TCTCCCAGCGTGCGCCAT; | (SEQ ID NO:131) |
| TCTGCCAGCGTGCGCCAT; | (SEQ ID NO:132) |
| TCTCCCAGZGTGZGCCAT; | (SEQ ID NO:133) |
| TCTTCGAA; TCTTCGAA; TCTTCTGCCCCCT | (SEQ ID NO:134) |
| GTGCA; | |
| TGACGTTTGACGTTTGACGTT; | (SEQ ID NO:135) |
| TGACTGTGAACGTTCGAGATGA; | (SEQ ID NO:136) |
| TGATCTTCCATCTATTAG; | (SEQ ID NO:137) |
| TGCACAGGGGGCAGAAGA; | (SEQ ID NO:138) |
| TGGTGGTGGTGGTGG; | (SEQ ID NO:139) |
| TTGCTTCGATCTTCGTCGTC; | (SEQ ID NO:140) |
| TTGGTGAAGGTAACGTTGAGGGGCAT. | (SEQ ID NO:141) |

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings, are not intended to be drawn to scale. In the drawings, each identical or nearly identical component that is illustrated in various figures is represented by a like numeral. For purposes of clarity, not every component may be labeled in every drawing. In the drawings:

FIG. 1 shows that shifting a CpG dinucleotide from a 5′ end to a 3′ end of an oligonucleotide results in decreased IFN-α production and a constant IL-10 stimulation: FIG. 1A shows IFN-α production in response to different oligonucleotides; FIG. 1B shows IL-10 production in response to different oligonucleotides;

FIG. 2 shows that oligonucleotides with strongly reduced IFN-α production result in optimal IL-10 stimulation when they contain an unmodified C in the CpG dinucleotide;

FIG. 3 shows that oligonucleotides with a higher T content result in higher IL-10 stimulation;

FIG. 4 shows that a 5′-TCG is required for efficient IFN-α production, whereas a 5′-TC is sufficient for potent IL-10 secretion;

FIG. 5 shows that IL-10 stimulation is maintained when the thymidine of the 5′-TC is chemically modified;

FIG. 6 shows that oligonucleotides with a 5′-TC or a 3′ shifted CpG dinucleotide induce stronger IL-10 production than oligonucleotides lacking a 5′-TC or a CpG;

FIG. 7 shows that oligonucleotides with a 5′-TC and a 3′ shifted CpG dinucleotide induce strong secretion of IL-6 or IL-10 but result in inefficient stimulation of cytokines or chemokines such as IFN-α or IP-10;

FIG. 8 shows that oligonucleotides with a 5′-TC and a 3′ shifted CpG efficiently induce the production of IL-6 and IL-10 from highly purified human B cells;

FIG. 9 shows that cells expressing the human TLR9 and an NFκB-Luciferase reporter are stimulated by oligonucleotides with a 5′-TC and a 3′ shifted CpG; and

FIG. 10 shows TLR9-mediated NFkB responses to oligonucleotides with CpG dinucleotides at different 3′ positions: FIG. 10A shows human cell responses; FIG. 10B shows murine cell responses.

### DETAILED DESCRIPTION

The invention provides CpG dinucleotide containing immunostimulatory nucleic acids that increase IL-10 expression without significantly increasing IFN-α expression. The nucleic acids of the invention are useful for treating diseases and disorders including autoimmune disorders.

In one aspect, the invention provides a nucleic acid, preferably an oligonucleotide, that includes a TC dinucleotide at its 5′ end and a CpG dinucleotide separated from the TC dinucleotide by at least two nucleotides.

In one embodiment, the CpG dinucleotide is separated from the TC dinucleotide by at least 2 nucleotides, and more preferably by 3, 4, 5, 6, 7, 8, 9, 10, 11, 12, 13, 14, 15, 16, 17, 18, 19, 20, 21, 22, 23, 25, 26, 27, 28, 29, or 30 or more nucleotides. In another embodiment, the CpG dinucleotide is included in the 3′ 80%, 75%, 70%, 65%, 60%, 55%, 50%, 45%, 40%, 35%, 30%, 25%, 20%, 15%, 10%, 5%, or 2.5% of the length of the nucleic acid molecule.

In some embodiments, the nucleic acid has two or more TC dinucleotides, two or more CpG dinucleotides, or combinations thereof. The 5′-most CpG dinucleotide is preferably separated from the 3′ most TC dinucleotide (which is 5′ to the 5′ most CpG dinucleotide) by 1, 2, 3, 4, 5, 6, 7, 8, 9, 10, 11, 12, 13, 14, 15, 16, 17, 18, 19, 20, 21, 22, 23, 25, 26, 27, 28, 29, or 30 or more nucleotides. The TC dinucleotides are preferably in the 5′ 10%, 20%, 30%, 40%, or 50% of the length of the nucleic acid. The CpG dinucleotides are in the 3′ 50%, 40%, 30%, 20%, or 10% of the length of the nucleic acid. However, the TC and CpG dinucleotides can be interspersed provided that there is a TC dinucleotide at the 5′ end of the molecule and that the 5′ most CpG is separated from the TC dinucleotide by 2, 3, 4, 5, 6, 7, 8, 9, 10, 11, 12, 13, 14, 15, 16, 17, 18, 19, 20, 21, 22, 23, 25, 26, 27, 28, 29, or 30 or more nucleotides, and the optimal distance between the 5′ TC and the CpG dinucleotide can depend on the length of the nucleic acid molecule. In preferred embodiments, the 3′ dinucleotide is preferably not a CpG dinucleotide.

In some embodiments, the 5′ dinucleotide is AC, GC, CC, TA, TG, or TT. However, a nucleic acid with a 5′ TC stimulates IL-10 production more effectively. In some embodiments, the nucleic acid has a modified C in the CpG dinucleotide. However, in other embodiments a nucleic acid with an unmodified C in the CpG dinucleotide can be used for ease of synthesis or to reduce potential in vivo toxicity.

Nucleic acids of the invention preferably have one or more stretches of poly T (e.g. 3T, 4T, 5T, 6T, 7T, 8T, 9T, 10T, or longer stretches of poly T). A preferred nucleic acid includes between 25% and 99%, preferably between 30% and 90%, preferably more than 35%, more than 40%, more than 45%, more than 50%, more than 55%, more than 60%, more than 65%, more than 70%, more than 75%, more than 80%, more than 85%, more than 90%, or more than 95% T nucleotides.

Preferred nucleic acids are between 5 and 100 nucleotides long, and preferably longer than about 10, 15, 20, 25, 30, 35, or 40 nucleotides long. However, longer nucleic acids are also embraced by the invention. A preferred nucleic acid is between about 10-20, 20-30, 30-40, 40-50, 50-60, 60-70, 70-80, 80-90, or 90-100 nucleotides long.

Preferred nucleic acids do not have a 5′ TCG trinucleotide. Nucleic acids can be provided as double-stranded molecules. Nucleic acids are preferably single-stranded molecules, and more preferably DNA molecules. However, one or more of the nucleotides and/or the internucleotide linkages can be modified as described herein.

In one embodiment, a nucleic acid of the invention has the following general formula:

5′ XYN₁YZN₂ 3′

wherein 5′ designates the 5′ end of the oligonucleotide and 3′ designates the 3′ end of the oligonucleotide, wherein X is a T or modified T nucleotide, wherein Y is a C or modified C nucleotide, wherein Z is a G or modified G nucleotide, wherein N₁ and N₂ are polynucleotides that do not include a CG dinucleotide, wherein N₁ does not include 5′ Z nucleotide, and wherein a 3′ polynucleotide consisting of the YZ dinucleotide and the N₂ polynucleotide contains a number of nucleotides that is at most 45% of the number of nucleotides in the oligonucleotide.

In another embodiment, a nucleic acid of the invention has the following general formula:

5′ XY N₁YZ N₂ 3′

wherein 5′ designates the 5′ end of the oligonucleotide and 3′ designates the 3′ end of the oligonucleotide, wherein X is a T or modified T nucleotide, wherein Y is a C or modified C nucleotide, wherein Z is a G or modified G nucleotide, wherein N₁ is a polynucleotide of 5 to 10 nucleotides, wherein N₁ does not include a CG dinucleotide, wherein N₁ does not include 5′ Z nucleotide, and wherein N₂ is a polynucleotide of 5 to 30 nucleotides;
Nucleic acids of the invention stimulate the production of IL-10 relative to that of IFN-α. The ratio of IL-10 induction relative to IFN-α induction is preferably between 1.5 and 10, and can be higher. In some embodiments, the ratio of induction is more than about 2.0, 2.5, 3.0, 3.5, 4.0, 4.5, 5.0, 5.5, 6.0, 6.5, 7.0, 7.5, 8.0, 8.5, 9.0, 9.5, or 10.0.

Immunostimulatory CpG nucleic acids of the invention form a subset of CpG nucleic acids that have distinct properties from immunostimulatory CpG nucleic acids previously studied. Three classes of CpG ODN have been described so far: the A-, B- and C-Classes. The most striking attribute of these described CpG ODN classes is their ability to stimulate the secretion of IFN-α from pDC and, therefore, of other effects that are mediated by type 1 interferons such as IP-10 production from monocytes (Blackwell (2003), J. Immunol. 170: 4061). Nevertheless, differences appear to exist between the stimulation of the two TLR9 expressing cells described to date: pDC and B cells (Uhlmann (2003), Current Drugs 6: 204). B cells are stimulated by immune modulatory ODN to secrete cytokines such as IL-6 or IL-10 (Krieg (2002), Annu. Rev. Immunol. 20:709). PDCs are, in contrast, stimulated to produce type I interferons. The CpG ODN classes described to date stimulate both PDC activation and cytokine production as well as B cell activation (Uhlmann (2003), Current Drugs 6: 204). However, the invention provides ODN sequences that stimulate few to no IFN-α secretion or related effects (such as IP-10 production from monocytes) but stimulate strong cytokine secretion from B cells in a TLR9-dependent way. The CpG immunostimulatory nucleic acids of the invention, termed T-Class ODN, lack a 5′-CG that is mainly responsible for the strong stimulatory effects mediated by CpG on human cells. In preferred embodiments, they contain a 5′TC that was shown to still retain potent and efficient cytokine production from B cells. In addition, such preferred ODN still bear a CpG dinucleotide, although in a more 3′ position. This CpG shift towards the 3′ end results in a strong decrease of pDC IFN-α production but not B cell IL-10 secretion. The CpG immunostimulatory nucleic acids of the invention induce efficient IL-10 production but don't induce efficient IFN-α production.

Although IL-10 is often considered to be a Th2-inducing cytokine, it can be a “suppressive” cytokine under certain conditions, for example when IL-10 production is out of proportion relative to other Th2 cytokines such as IL-4, IL-5, and IL-13. Studies demonstrated that IL-10 is involved in the reduction of inflammatory responses and autoimmune diseases (Mocellin (2003), TRENDS 24: 36). This effect involves regulatory lymphocytes, T cells as well as B cells (Shevach (2002), Nature Reviews Immunol. 2: 389; Sakaguchi (2003), Nature Immunol. 4: 10; Fillatreau (2002), Nature Immunol. 10: 944; Mauri (2003), J. Exp. Med. 197: 489; Mizoguchi (2002), Immunity 16: 219). IL-10 was demonstrated in vitro to be responsible for the generation of IL-10 producing regulatory T cells (Shevach (2002), Nature Reviews Immunol. 2: 389). These T cells appear to influence the immune response of the host to e.g. bacterial infections. These T cells were also demonstrated to help to protect from autoimmune disease development (Shevach (2002), Nature Reviews Immunol. 2: 389). The same effect was observed with regulatory B cells (Fillatreau (2002), Nature Immunol. 10: 944; Mauri (2003), J. Exp. Med. 197: 489; Mizoguchi (2002), Immunity 16: 219). In one embodiment of the invention, T-class CpG ODN are used to mediate strong stimulation of B cells that produce high levels of IL-10, and are useful as therapy for autoimmune diseases.

In one aspect, CpG stimulatory nucleic acids of the invention are useful to induce increased IL-10 levels in relation to IFN-α levels. In one embodiment, the ratio of IL-10/IFN-α expression induced by an oligonucleotide of the invention is at least 50% higher than the ratio of IL-10/IFN-α expression induced by a reference oligonucleotide, for example:

| | |
|---|---|
| 5′ T*C*G*T*C*G*T*T*T*T*G*T*C*G*T* | (SEQ ID NO:54) |
| T*T*T*G*T*C*G*T*T 3′, | |
| 5′ T*C*G*T*C*G*T*T*T*T*G*T*C*G*T* | (SEQ ID NO:142) |
| T*T*T*T*T*T*C*G*A 3′, | |
| or | |
| 5′ T*C*G*T*C*G*T*T*T*C*G*T*C*G*T* | (SEQ ID NO:143) |
| T*T*T*G*T*C*G*T*T 3′. | |

The ratio may be even higher, e.g., 2 fold, 3 fold, 4 fold, 5 fold, 10 fold, 50 fold, 100 fold, or more. The ratio of IL-10/IFN-α induced by an oligonucleotide may be calculated by dividing the induced amount or percent of IL-10 increase by the induced amount or percent of IFN-α increase. The induced amount or percent increase of expression of a molecule may be calculated by comparing the expression levels of the molecule before and after treatment with the oligonucleotide. The expression levels may be RNA or protein expression levels.

In one embodiment, an oligonucleotide of the invention induces an increase in IL-10 expression that is similar to that of a reference oligonucleotide (e.g., one of the reference oligonucleotides described above). However, the induced increase in IFN-α expression may be significantly lower (e.g., 2 fold, 3, fold, 4 fold, 5 fold, 10 fold, or 50 fold lower, etc.) than that obtained with the reference oligonucleotide. This results in a higher ratio of IL-10/IFN-α induction using an oligonucleotide of the invention. In one embodiment, only background levels of IFN-α are obtained with an immunostimulatory nucleic acid of the invention.

However, in other embodiments, the absolute level of IL-10 induction obtained with an oligonucleotide of the invention is higher than that obtained with a reference oligonucleotide (e.g., 50% more, 2 fold, 3 fold, 4 fold, 5 fold, 10 fold, or 50 fold higher, etc.).

Accordingly, in one aspect of the invention, T-class CpG stimulatory nucleic acids are used to stimulate IL-10 production. In some embodiments, the CpG stimulatory nucleic acids indirectly stimulate IL-10 production from macrophages. In other embodiments, the CpG stimulatory nucleic acids stimulate IL-10 production from B cells. In yet further embodiments, the CpG stimulatory nucleic acids stimulate IL-10 production from one or more cell types. IL-10 production in the absence of IFN-α production is useful to treat diseases and conditions such as autoimmune diseases or disorders. In some embodiments, IL-10 production is useful to activate T regulatory cells. In other embodiments, IL-10 production is useful to activate B regulatory cells. In yet further embodiments, IL-10 production is useful to suppress Th1 cytokines. IL-10 production can be particularly useful to treat a subject with, or at risk of developing, one or more Th2-mediated allergic diseases or disorders. IL-10 can also be used to control autoimmune diseases such as autoimmune encephalomyelitis. Autoimmune diseases include, but are not limited to, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

In another aspect, CpG stimulatory nucleic acids of the invention are useful to stimulate a regulatory T cell response. Regulatory T cells can control diseases such as inflammatory bowel disease and are involved in the control of other immune responses including autoimmune responses.

Regulatory T cell activation can be used to regulate antibody specific responses, particularly in the context of allergies and autoimmune diseases. In some embodiments, the CpG immunostimulatory nucleic acids are used for treating and preventing antibody-mediated autoimmune diseases. In some autoimmune diseases, a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus. Some of these autoimmune diseases can also associated with organ-specific autoimmune disorders involving a Th2 response.

In some embodiments, antigen-specific regulatory T cell responses can be stimulated by administering a specific antigen, preferably a self-antigen, along with (not long before, simultaneously, or not long after) an immunostimulatory CpG nucleic acid of the invention. In some instances, the CpG immunostimulatory nucleic acids are delivered with low doses of self-antigens.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer.

In yet another aspect, CpG immunostimulatory nucleic acids of the invention are used to stimulate a regulatory B cell response. The stimulation of regulatory B cells can be used to control diseases such as autoimmune disorders. In some embodiments, antigen-specific regulatory B cell responses can be stimulated by administering a specific antigen before, with, or after an immunostimulatory CpG nucleic acid of the invention. In some embodiments, Th2-mediated diseases such as asthma and allergy can be treated by administering one or more CpG immunostimulatory nucleic acids of the invention with one or more allergens. In another embodiment, SLE can be treated by administering one or more CpG stimulatory nucleic acids of the invention with one or more antigens such as purified components of nucleosomes or ribonucleoproteins. In a further embodiment, rheumatoid arthritis can be treated by administering one or more CpG stimulatory nucleic acids of the invention with one or more antigens such as an immunoglobulin.

In a further aspect, CpG stimulatory nucleic acids of the invention are used to stimulate a T regulatory response. These nucleic acids can be administered (e.g. as an adjuvant for vaccines or as a monotherapy) in a number of diseases for which strong T regulatory responses might be more important such as Crohn's disease, allograft rejection or spontaneous abortion (McCluskie (2001), Vaccine 19: 413). In some embodiments, the CpG stimulatory nucleic acids of the invention are administered mucosally. Examples of mucosal administration methods and formulations are disclosed in (U.S. Patent Publication 20010044416), the entire disclosure of which is incorporated herein by reference.

Stimulation of a T regulatory response can be useful to treat certain autoimmune diseases and conditions such as organ specific autoimmune disorders (e.g. Crohn's disease, peptic ulcer, acute solid organ allograft rejection, and unexplained recurrent abortion). Stimulation of a T regulatory response can also be useful to induce an antigen-specific response by administering an antigen to a subject along with a nucleic acid of the invention in an amount effective to produce an antigen-specific immune response.

According to the invention, the terms “nucleic acid” and “oligonucleotide” also encompass nucleic acids or oligonucleotides with substitutions or modifications, such as in the bases and/or sugars. For example, they include nucleic acids having backbone sugars that are covalently attached to low molecular weight organic groups other than a hydroxyl group at the 2′ position and other than a phosphate group or hydroxy group at the 5′ position. Thus modified nucleic acids may include a 2′-O-alkylated ribose group. In addition, modified nucleic acids may include sugars such as arabinose or 2′-fluoroarabinose instead of ribose. Thus the nucleic acids may be heterogeneous in backbone composition thereby containing any possible combination of polymer units linked together such as peptide-nucleic acids (which have an amino acid backbone with nucleic acid bases).

Nucleic acids also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, thymine, 5-methylcytosine, 5-hydroxycytosine, 5-fluorocytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

The immunostimulatory oligonucleotides of the instant invention can encompass various chemical modifications and substitutions, in comparison to natural RNA and DNA, involving a phosphodiester internucleotide bridge, a β-D-ribose unit and/or a natural nucleotide base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleotide bridge and/or at a particular β-D-ribose unit and/or at a particular natural nucleotide base position in comparison to an oligonucleotide of the same sequence which is composed of natural DNA or RNA.

For example, the invention relates to an oligonucleotide which may comprise one or more modifications and wherein each modification is independently selected from:
a) the replacement of a phosphodiester internucleotide bridge located at the 3′ and/or the 5′ end of a nucleotide by a modified internucleotide bridge,
b) the replacement of phosphodiester bridge located at the 3′ and/or the 5′ end of a nucleotide by a dephospho bridge,
c) the replacement of a sugar phosphate unit from the sugar phosphate backbone by another unit,
d) the replacement of a β-D-ribose unit by a modified sugar unit, and
e) the replacement of a natural nucleotide base by a modified nucleotide base.

More detailed examples for the chemical modification of an oligonucleotide are as follows.

A phosphodiester internucleotide bridge located at the 3′ and/or the 5′ end of a nucleotide can be replaced by a modified internucleotide bridge, wherein the modified internucleotide bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)-O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some preferred embodiments the sugar is 2′-O-methylribose, particularly for one or both nucleotides linked by a phosphodiester or phosphodiester-like internucleotide linkage.

Nucleic acids also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleotide base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleotides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleotide bases. This list is meant to be exemplary and is not to be interpreted to be limiting.

In particular formulas described herein a set of modified bases is defined. For instance the letter Y is used to refer to a nucleotide containing a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

The letter Z is used to refer to guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C₂-C₆)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′3′-linkage may be a phosphodiester, phosphorothioate or any other modified internucleotide bridge. Methods for accomplishing such linkages are known in the art. For instance, such linkages have been described in Seliger, H.; et al., Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′3′-linked nucleic acids where the linkage between the 3′-terminal nucleotides is not a phosphodiester, phosphorothioate or other modified bridge, can be prepared using an additional spacer, such as tri- or tetra-ethylenglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

It recently has been reported that CpG oligonucleotides appear to exert their immunostimulatory effect through interaction with Toll-like receptor 9 (TLR9). Hemmi H et al. (2000) Nature 408: 740-5. TLR9 signaling activity thus can be measured in response to CpG oligonucleotide or other immunostimulatory nucleic acid by measuring NF-κB, NF-κB-related signals, and suitable events and intermediates upstream of NF-κB.

For use in the instant invention, the oligonucleotides of the invention can be synthesized de novo using any of a number of procedures well known in the art. For example, the b-cyanoethyl phosphoramidite method (Beaucage, S. L., and Caruthers, M. H., Tet. Let. 22:1859, 1981); nucleotide H-phosphonate method (Garegg et al., Tet. Let. 27:4051-4054, 1986; Froehler et al., Nucl. Acid. Res. 14:5399-5407, 1986,; Garegg et al., Tet. Let. 27:4055-4058, 1986, Gaffney et al., Tet. Let. 29:2619-2622, 1988). These chemistries can be performed by a variety of automated nucleic acid synthesizers available in the market. These oligonucleotides are referred to as synthetic oligonucleotides. An isolated oligonucleotide generally refers to an oligonucleotide which is separated from components which it is normally associated with in nature. As an example, an isolated oligonucleotide may be one which is separated from a cell, from a nucleus, from mitochondria or from chromatin.

The oligonucleotides are partially resistant to degradation (e.g., are stabilized). A “stabilized oligonucleotide molecule” shall mean an oligonucleotide that is relatively resistant to in vivo degradation (e.g. via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases. Other modified oligonucleotides include phosphodiester modified nucleic acids, combinations of phosphodiester and phosphorothioate nucleic acid, methylphosphonate, methylphosphorothioate, phosphorodithioate, p-ethoxy, and combinations thereof.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1:165, 1990).

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

As described herein, the oligonucleotides of the invention may have phosphodiester or phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and S_{P} stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rp may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

A surprisingly strong effect is achieved by the p-chirality at the CpG dinucleotide itself. In comparison to a stereo-random CpG oligonucleotide the congener in which the single CpG dinucleotide was linked in Rp was slightly more active, while the congener containing an Sp linkage was nearly inactive for inducing spleen cell proliferation.

According to the invention, a subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to treat cancer and tumors, infections, and allergy/asthma in non human subjects. Cancer is one of the leading causes of death in companion animals (i.e., cats and dogs).

As used herein, the term treat, treated, or treating when used with respect to an disorder such as an infectious disease, cancer, allergy, or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

In the instances when the CpG oligonucleotide is administered with an antigen, the subject may be exposed to the antigen. As used herein, the term exposed to refers to either the active step of contacting the subject with an antigen or the passive exposure of the subject to the antigen in vivo. Methods for the active exposure of a subject to an antigen are well-known in the art. In general, an antigen is administered directly to the subject by any means such as intravenous, intramuscular, oral, transdermal, mucosal, intranasal, intratracheal, or subcutaneous administration. The antigen can be administered systemically or locally. Methods for administering the antigen and the CpG immunostimulatory nucleic acid are described in more detail below. A subject is passively exposed to an antigen if an antigen becomes available for exposure to the immune cells in the body. A subject may be passively exposed to an antigen, for instance, by entry of a foreign pathogen into the body or by the development of a tumor cell expressing a foreign antigen on its surface.

The methods in which a subject is passively exposed to an antigen can be particularly dependent on timing of administration of the CpG immunostimulatory nucleic acid. For instance, in a subject at risk of developing a cancer or an infectious disease or an allergic or asthmatic response, the subject may be administered the CpG immunostimulatory nucleic acid on a regular basis when that risk is greatest, i.e., during allergy season or after exposure to a cancer causing agent. Additionally the CpG immunostimulatory nucleic acid may be administered to travelers before they travel to foreign lands where they are at risk of exposure to infectious agents. Likewise the CpG immunostimulatory nucleic acid may be administered to soldiers or civilians at risk of exposure to biowarfare to induce a systemic or mucosal immune response to the antigen when and if the subject is exposed to it.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and muticellular organisms such as parasites and allergens. The term antigen broadly includes any type of molecule which is recognized by a host immune system as being foreign. Antigens include but are not limited to cancer antigens, microbial antigens, and allergens.

In methods of the invention, the CpG immunostimulatory nucleic acids may be directly administered to the subject or may be administered in conjunction with a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell. Examples of nucleic acid delivery complexes include nucleic acids associated with a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the oligonucleotide is released in a functional form.

Delivery vehicles or delivery devices for delivering antigen and oligonucleotides to surfaces have been described. The CpG immunostimulatory nucleic acid and/or the antigen and/or other therapeutics may be administered alone (e.g., in saline or buffer) or using any delivery vehicles known in the art. For instance the following delivery vehicles have been described: Cochleates; Emulsomes; ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Nucleic acid vaccines; Polymers (e.g. carboxymethylcellulose, chitosan); Polymer rings; Proteosomes; Sodium Fluoride; Transgenic plants; Virosomes; Virus-like particles. Other delivery vehicles are known in the art and some additional examples are provided herein.

The term effective amount of a CpG immunostimulatory nucleic acid refers to the amount necessary or sufficient to realize a desired biologic effect. For example, an effective amount of a CpG immunostimulatory nucleic acid administered with an antigen for inducing mucosal immunity is that amount necessary to cause the development of IgA in response to an antigen upon exposure to the antigen, whereas that amount required for inducing systemic immunity is that amount necessary to cause the development of IgG in response to an antigen upon exposure to the antigen. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular CpG immunostimulatory nucleic acid being administered the size of the subject, or the severity of the disease or condition. One of ordinary skill in the art can empirically determine the effective amount of a particular CpG immunostimulatory nucleic acid and/or antigen and/or other therapeutic agent without necessitating undue experimentation.

Subject doses of the compounds described herein for mucosal or local delivery typically range from about 0.1 μg to 10 mg per administration, which depending on the application could be given daily, weekly, or monthly and any other amount of time therebetween. More typically mucosal or local doses range from about 10 μg to 5 mg per administration, and most typically from about 100 μg to 1 mg, with 2-4 administrations being spaced days or weeks apart. More typically, immune stimulant doses range from 1 μg to 10 mg per administration, and most typically 10 μg to 1 mg, with daily or weekly administrations. Subject doses of the compounds described herein for parenteral delivery for the purpose of inducing an antigen-specific immune response, wherein the compounds are delivered with an antigen but not another therapeutic agent are typically 5 to 10,000 times higher than the effective mucosal dose for vaccine adjuvant or immune stimulant applications, and more typically 10 to 1,000 times higher, and most typically 20 to 100 times higher. Doses of the compounds described herein for parenteral delivery for the purpose of inducing an innate immune response or for increasing ADCC or for inducing an antigen specific immune response when the CpG immunostimulatory nucleic acids are administered in combination with other therapeutic agents or in specialized delivery vehicles typically range from about 0.1 μg to 10 mg per administration, which depending on the application could be given daily, weekly, or monthly and any other amount of time therebetween. More typically parenteral doses for these purposes range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

For any compound described herein the therapeutically effective amount can be initially determined from animal models. A therapeutically effective dose can also be determined from human data for CpG oligonucleotides which have been tested in humans (human clinical trials have been initiated) and for compounds which are known to exhibit similar pharmacological activities, such as other adjuvants, e.g., LT and other antigens for vaccination purposes. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the CpG immunostimulatory nucleic acid can be administered to a subject by any mode that delivers the oligonucleotide to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

For oral administration, the compounds (i.e., CpG immunostimulatory nucleic acids, antigens and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the oligonucleotide (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression. Colorants and flavoring agents may all be included. For example, the oligonucleotide (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the oligonucleotide or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the oligonucleotides (or derivatives thereof). The oligonucleotide (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (al-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent® nebulizer, manufactured by Mallinckrodt, Inc., St. Louis, Mo.; the Acorn II nebulizer, manufactured by Marquest Medical Products, Englewood, Colo.; the Ventolin® metered dose inhaler, manufactured by Glaxo Inc., Research Triangle Park, N.C.; and the Spinhaler® powder inhaler, manufactured by Fisons Corp., Bedford, Mass.

All such devices require the use of formulations suitable for the dispensing of oligonucleotide (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified oligonucleotide may also be prepared in different formulations depending on the type of chemical modification or the type of device employed.

Formulations suitable for use with a nebulizer, either jet or ultrasonic, will typically comprise oligonucleotide (or derivative) dissolved in water at a concentration of about 0.1 to 25 mg of biologically active oligonucleotide per mL of solution. The formulation may also include a buffer and a simple sugar (e.g., for oligonucleotide stabilization and regulation of osmotic pressure). The nebulizer formulation may also contain a surfactant, to reduce or prevent surface induced aggregation of the oligonucleotide caused by atomization of the solution in forming the aerosol.

Formulations for use with a metered-dose inhaler device will generally comprise a finely divided powder containing the oligonucleotide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing oligonucleotide (or derivative) and may also include a bulking agent, such as lactose, sorbitol, sucrose, or mannitol in amounts which facilitate dispersal of the powder from the device, e.g., 50 to 90% by weight of the formulation. The oligonucleotide (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The CpG immunostimulatory nucleic acids and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a CpG immunostimulatory nucleic acid and optionally antigens and/or other therapeutic agents optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

In some embodiments, an immunostimulatory oligonucleotide of the invention can be linked to one or more lipophilic groups (L).

A lipophilic group L is preferably a cholesteryl or modified cholesteryl residue. The cholesterol moiety may be reduced (e.g. as in cholestan) or may be substituted (e.g. by halogen). A combination of different lipophilic groups in one molecule is also possible. Other lipophilic groups include but are not limited to bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

In some embodiments, L is preferably at or near the 3′ end of an oligonucleotide. L may be connected to the oligonucleotide by a linker moiety. Optionally the linker moiety is a non-nucleotidic linker moiety. Non-nucleotidic linkers are e.g. abasic residues (dSpacer), oligoethyleneglycol, such as triethyleneglycol (spacer 9) or hexaethylenegylcol (spacer 18), or alkane-diol, such as butanediol. The spacer units are preferably linked by phosphodiester or phosphorothioate bonds. The linker units may appear just once in the molecule or may be incorporated several times, e.g. via phosphodiester, phosphorothioate, methylphosphonate, or amide linkages.

The lipophilic group L may be attached at various positions of an oligonucleotide. In some embodiments, the lipophilic group L is linked to the 3′-end of the oligonucleotide, where it also serves the purpose to enhance the stability of the oligomer against 3′-exonucleases. Alternatively, it may be linked to an internal nucleotide or a nucleotide on a branch. The lipophilic group L may be attached to a 2′-position of the nucleotide. The lipophilic group L may also be linked to the heterocyclic base of the nucleotide.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

Materials and Methods:

Oligodeoxynucleotides:

All ODN were purchased from Biospring (Frankfurt, Germany), controlled for identity and purity by Coley Pharmaceutical Group (Langenfeld, Germany) and had undetectable endotoxin levels (<0.1 EU/ml) measured by the Limulus assay (BioWhittaker, Verviers, Belgium). ODN were suspended in sterile, endotoxin-free Tris-EDTA (Sigma, Deisenhofen, Germany), and stored and handled under aseptic conditions to prevent both microbial and endotoxin contamination. All dilutions were carried out using pyrogen-free phosphate-buffered saline (Life Technologies, Eggenstein, Germany).

The following table shows the sequences of the oligonucleotides (shown 5′ to 3′) used in the following experiments (* is a phosphorothioate, and _is a phosphodiester or phosphodiester like).

| | | |
|---|---|---|
| SEQ ID NO:1 | T*C*T*T*T*T*T*T*T*T*T*T*T*T*T*T*T | |
| SEQ ID NO:2 | T*C*T*T*T*T*T*T*T*T*C*G*T*T*T*T*T | 5′-TC + CpG 3′ |
| SEQ ID NO:3 | T*C*T*T*T*T*T*T*T*T*T*T*T*C*G*T*T | 5′-TC + CpG 3′ |
| SEQ ID NO:4 | T*C*T*T*T*T*T*T*G*T*C*G*T*T*T*T*T | 5′-TC + CpG 3′ |
| SEQ ID NO:5 | T*C*T*T*T*T*T*T*T*T*T*G*T*C*G*T*T | 5′-TC + CpG 3′ |
| SEQ ID NO:6 | T*C*T*T*T*T*T*T*T*T*C*G*T*T*T*T*T*T*T*T*T*T | 5′-TC + CpG 3′ |
| SEQ ID NO:7 | T*C*T*T*T*T*T*T*T*T*T*T*T*C*G*T*T*T*T*T*T*T | 5′-TC + CpG 3′ |
| SEQ ID NO:8 | T*C*T*T*T*T*T*T*T*T*T*T*T*T*T*C*G*T*T*T*T*T | 5′-TC + CpG 3′ |
| SEQ ID NO:9 | T*C*T*T*T*T*T*T*G*T*C*G*T*T*T*T*T*T*T*T*T*T | 5′-TC + CpG 3′ |
| SEQ ID NO:10 | T*C*T*T*T*T*T*T*T*T*T*G*T*C*G*T*T*T*T*T*T*T | 5′-TC + CpG 3′ |
| SEQ ID NO:11 | T*C*T*T*T*T*T*T*T*T*T*T*T*G*T*C*G*T*T*T*T*T | 5′-TC + CpG 3′ |
| SEQ ID NO:12 | T*C*T*T*T*T*T*T_T*T*C*G*T*T*T*T*T | 5′-TC + CpG 3′ TTCG w/ PO bond |
| SEQ ID NO:13 | T*C*T*T*T*T*T*T_T*T*C*G*T*T*T*T*T*T*T*T*T*T | 5′-TC + CpG 3′ TTCG w/ PO bond |
| SEQ ID NO:14 | T*C*T*T*T*T_T*T*C*G*T*T*T*T*T*T*T*T*T*T*T*T | 5′-TC + CpG 3′ TTCG w/ PO bond |
| SEQ ID NO:15 | T*C*C*A*G*G*A*C*T*T*C*T*C*T*C*A*G*G*T*T | |
| SEQ ID NO:16 | G*C*C*A*G*G*A*C*T*T*C*T*C*T*C*A*G*G*T*T | 5′-GC |
| SEQ ID NO:17 | T*C*C*A*T*T*A*C*T*T*C*T*C*T*C*A*T*T*T*T | GG to TT |
| SEQ ID NO:18 | T*C*C*A*G*G*A*T*C*T*C*T*C*T*C*A*G*G*T*T | CT to TC |
| SEQ ID NO:19 | T*C*C*A*G*G*A*C*T*T*G*T*G*T*G*A*G*G*T*T | TC to TG |
| SEQ ID NO:20 | G*C*C*A*G*G*A*C*A*C*C*T*C*A*C*A*G*G*A*T | 5′-GC and T to A |
| SEQ ID NO:21 | T*C*T*T*T*T*T*T*C*T*T*T*C*T*T*T*T | TC ODN |
| SEQ ID NO:22 | T*C*T*T*C*T*T*T*T*T*T*T*T*T*T*T*T | TC ODN |
| SEQ ID NO:23 | T*C*T*T*T*T*T*C*T*T*C*T*C*T*C*T*T*T*T*T | |
| SEQ ID NO:24 | T*C*T*T*T*T*T*T*G*T*C_G*T*T*T*T*T*T*T*T*T*T | |
| SEQ ID NO:25 | T*C_T*T*T*T*T*T*G*T*C*G*T*T*T*T*T*T*T*T*T*T | |
| SEQ ID NO:26 | T*C_T*T*T*T*T*T*G*T*C_G*T*T*T*T*T*T*T*T*T*T | |
| | T*C*T*T*T*T*T*T*T*T*T*T*T*T*T*T*T*T*T*T*T*T | |
| SEQ ID NO:27 | *T*T | 24 mer |
| SEQ ID NO:28 | T*A*T*T*T*T*T*T*T*T*T*T*T*T*T*T*T | 5′-TA |
| SEQ ID NO:29 | T*G*T*T*T*T*T*T*T*T*T*T*T*T*T*T*T | 5′-TG |
| SEQ ID NO:30 | T*Z*T*T*T*T*T*T*T*T*T*T*T*T*T*T*T | 5′-TZ |
| SEQ ID NO:31 | U*C*T*T*T*T*T*T*T*T*T*T*T*T*T*T*T | 5′-UC |
| SEQ ID NO:32 | 5T*C*T*T*T*T*T*T*T*T*T*T*T*T*T*T*T | 5T: 5-Methoxythymidine |
| SEQ ID NO:33 | T*5H*T*T*T*T*T*T*T*T*T*T*T*T*T*T*T | 5H: 5-Hydroxy-deoxycytidine |
| SEQ ID NO:34 | T*C*G*A*A*A*A*A*A*A*A*A*A*T*A*A*A | poly A + 5′ TCG increasing T amount |
| SEQ ID NO:35 | T*C*G*A*A*A*A*A*A*A*A*A*T*T*A*A*A | poly A + 5′ TCG increasing T amount |
| SEQ ID NO:36 | T*C*G*A*A*A*A*A*A*A*T*T*T*T*A*A*A | poly A + 5′ TCG increasing T amount |
| SEQ ID NO:37 | T*C*G*A*A*A*A*A*T*T*T*T*T*T*A*A*A | poly A + 5′ TCG increasing T amount |
| SEQ ID NO:38 | T*C*G*A*A*A*T*T*T*T*T*T*T*T*T*T*A | poly A + 5′ TCG increasing T amount |
| SEQ ID NO:39 | T*C*G*T*A*A*A*A*A*A*A*A*A*A*A*A*A | poly A + 5′ TCG increasing T amount |
| SEQ ID NO:40 | T*C*G*T*T*T*A*A*A*A*A*A*A*A*A*A*A | poly A + 5′ TCG increasing T amount |
| SEQ ID NO:41 | T*C*G*A*A*A*A*A*A*A*A*A*A*A*A*A*A | poly A + TCG 5′ |
| SEQ ID NO:42 | T*C*G*T*T*T*T*T*T*T*T*T*T*T*T*T*T | 1x TCG 5′ + poly T |
| SEQ ID NO:43 | T*T*T*T*T*T*T*T*T*T*T*T*T*T*T*T*T | |
| SEQ ID NO:44 | T*T*T*T*T*T*T*T*T*T*T*T*T*T*T*C*G | poly T + TCG 3′ |
| SEQ ID NO:45 | T*T*T*C*G*T*T*T*T*T*T*T*T*T*T*T*T | poly T + CG various positions |
| SEQ ID NO:46 | T*T*T*T*T*T*C*G*T*T*T*T*T*T*T*T*T | poly T + CG various positions |
| SEQ ID NO:47 | T*T*T*T*T*T*T*T*T*C*G*T*T*T*T*T*T | poly T + CG various positions |
| SEQ ID NO:48 | T*T*C*G*T*T*T*T*T*T*T*T*T*T*T*T*T | CG shift |
| SEQ ID NO:49 | T*T*T*T*C*G*T*T*T*T*T*T*T*T*T*T*T | CG shift |
| SEQ ID NO:50 | T*T*T*T*T*C*G*T*T*T*T*T*T*T*T*T*T | poly T + CG ODN 5xT 5′ |
| | T*T*T*T*T*T*T*T*T*C*G*T*T*T*T*T*T*T*T*T*T*T | |
| SEQ ID NO:51 | *T | 24 mer |
| SEQ ID NO:52 | T*T*T*T*T*T*.T*T*T*Z*G*T*T*T*T*T*T | ZpG |
| SEQ ID NO:53 | A*T*T*T*T*T*T*T*T*C*G*T*T*T*T*T*T | 5′ A |
| | T*C*G*T*C*G*T*T*T*T*G*T*C*G*T*T*T*T*G*T*C*G | |
| SEQ ID NO:54 | *T*T | |
| SEQ ID NO:55 | T*C*G*C*C*C*C*C*C*C*C*C*C*C*C*C*C | |
| SEQ ID NO:56 | A*C*G*T*T*T*T*T*T*T*T*T*T*T*T*T*T | |
| SEQ ID NO:57 | C*C*G*T*T*T*T*T*T*T*T*T*T*T*T*T*T | |
| SEQ ID NO:58 | G*C*G*T*T*T*T*T*T*T*T*T*T*T*T*T*T | |
| SEQ ID NO:59 | T*T*G*T*T*T*T*T*T*T*T*T*T*T*T*T*T | |
| SEQ ID NO:60 | T*C*G*T*C*G*T*T*T*T*C*G*G*C*G*C*G*C*G*C*C*G | |
| SEQ ID NO:61 | T*C*C*A*T*G*A*C*G*T*T*C*C*T*G*A*C*G*T*T | |

TLR9 Assays:

Stably transfected HEK293 cells expressing the human or mouse TLR9 were described before. Briefly, HEK293 cells were transfected by electroporation with vectors expressing the human or mouse TLR9 and a 6×NFκB-luciferase reporter plasmid. Stable transfectants (3×10⁴ cells/well) were incubated with ODN for 16 h at 37° C. in a humidified incubator. Each data point was done in triplicate. Cells were lysed and assayed for luciferase gene activity (using the BriteLite kit from Perkin-Elmer, Zaventem, Belgium). Stimulation indices were calculated in reference to reporter gene activity of medium without addition of ODN.

Cell Purification:

Peripheral blood buffy coat preparations from healthy human donors were obtained from the Blood Bank of the University of Düsseldorf (Germany) and PBMC were purified by centrifugation over Ficoll-Hypaque (Sigma). Cells were cultured in a humidified incubator at 37° C. in RPMI 1640 medium supplemented with 5% (v/v) heat inactivated human AB serum (BioWhittaker) or 10% (v/v) heat inactivated FCS, 2 mM L-glutamine, 100 U/ml penicillin and 100 μg/ml streptomycin (all from Sigma).

Cytokine Detection:

PBMC were resuspended at a concentration of 3×10⁶ cells/ml and added to 48 well flat-bottomed plates (1 ml/well) or 96 well round-bottomed plates (250 μl/well). PBMC were incubated with various ODN concentrations and culture supernatants (SN) were collected after the indicated time points. If not used immediately, SN were frozen at −20° C. until required.

Amounts of cytokines in the SN were assessed using commercially available ELISA Kits (IL-6, IP-10, IFN-γ or IL-10; from Diaclone, Besançon, France) or an in-house ELISA for IFN-α developed using commercially available antibodies (from PBL, New Brunswick, N.J., USA for detection of multiple IFN-α species).

Isolation of Human B Cells:

Human B cells were isolated from whole PBMC with the CD19 B cell isolation kit as described by the manufacturer (Miltenyi, Bergisch-Gladbach, Germany). To determine purity cells were stained with mAb to CD20 and CD14 and cells identified by flow cytometry. In all experiments B cells were more than 95% pure. Purified B cells (2×10⁵ to 5×10⁵ cells/ml) were incubated with increasing concentrations of ODN for 24 h and IL-6 or IL-10 measured as described above.

### Example 1

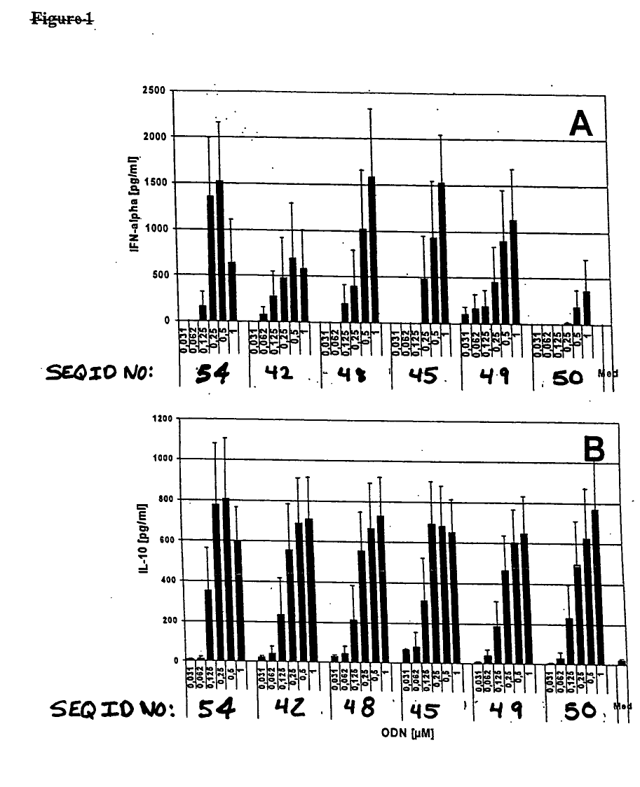

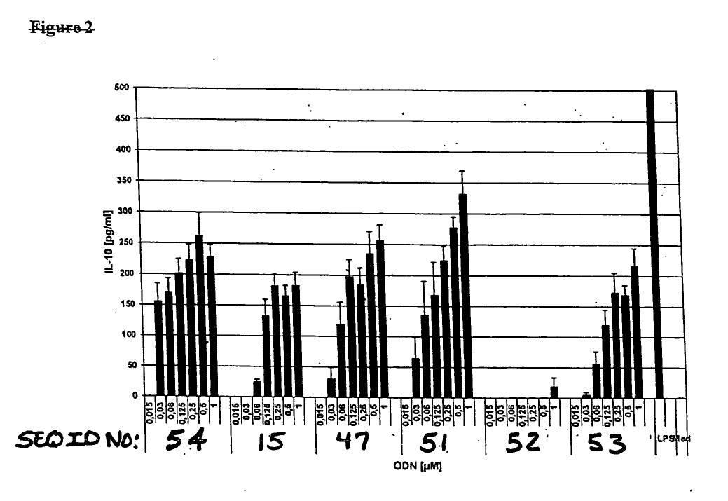

By shifting the immunostimulatory CpG dinucleotide in a phosphorothioate ODN from the 5′ end to the 3′ end, a graded decrease of IFN-α production was observed while retaining IL-10 stimulation. Human PBMC were incubated with increasing concentrations of the indicated ODN for 48 h. SN were harvested and IFN-α (A) and IL-10 (B) measured by ELISA. FIG. 1 shows the Mean±SEM of three donors for each experimental condition.

The data demonstrate that although the production of IFN-α decreases with ODNs containing a CpG dinucleotide shifted toward the 3′ end, the level of IL-10 secretion remains relatively constant. Therefore, a 5′CpG location causes IFN-α production. Shifting the CpG dinucleotide to the 3′ end does not result in loss of immune stimulation, only in loss of efficient IFN-α secretion.

### Example 2

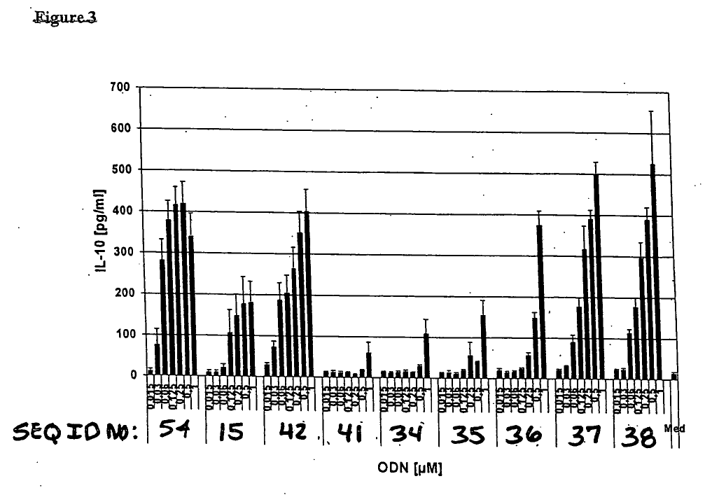

Human PBMC were incubated with increasing concentrations of the indicated ODN for 48 h. SN were harvested and IL-10 measured by ELISA. FIG. 2 shows the Mean±SEM of three donors for each experimental condition.

The data demonstrate that for ODNs with a 3′ shifted CpG dinucleotide, the cytosine has to be 5-unmethylated for efficient IL-10 induction. In addition, increasing the length of the ODN appears to result in enhanced IL-10 stimulation (SEQ ID NO: 51).

### Example 3

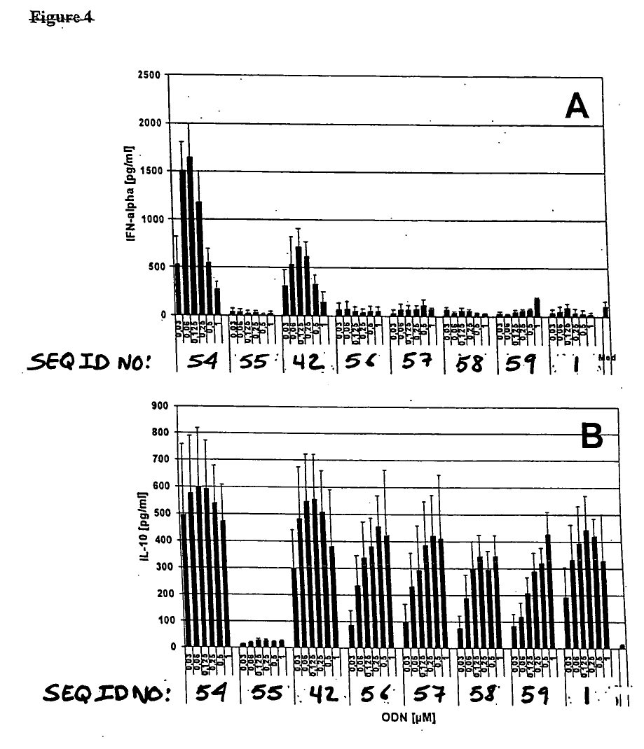

The T content of an ODN determines its immune stimulatory activity. Human PBMC were incubated with the indicated concentrations of ODN with decreasing T content for 48 h. SN were harvested and IL-10 measured by ELISA. FIG. 3 shows the Mean±SEM of three donors for each experimental condition.

The data demonstrate that the content of thymidine nucleobases in a phosphorothioate ODN determines its capacity to induce IL-10 production. An ODN with a 5′-TCG and an increasing number of adenosine nucleotides looses its capacity to efficiently stimulate IL-10 production. Therefore, a certain thymidine content is required for efficient IL-10 production.

### Example 4

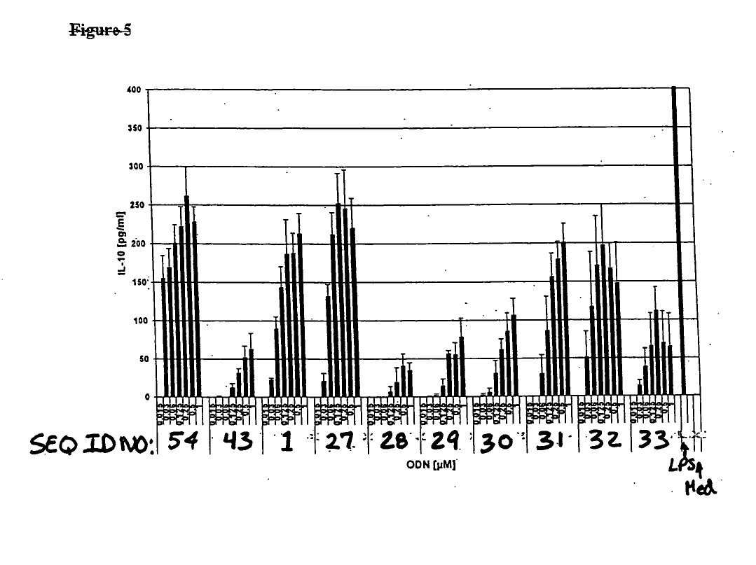

A 5′-TCG is required for efficient IFN-α production, whereas a 5′-TC is sufficient for potent IL-10 secretion. Human PBMC were incubated with increasing concentrations of the indicated ODN for 48 h. SN were harvested and IFN-α (A) and IL-10(B) measured by ELISA. FIG. 4 shows the Mean±SEM of three donors for each experimental condition.

The data demonstrate that a 5′-TCG in a phosphorothioate ODN is required to induce efficient IFN-α secretion. All other 5′ trinucleotides (5′-ACG, CCG or GCG) do not appear to have an effect on type I interferon secretion. In addition, exchange of the 5′-CG to 5′-TG or 5′-CT (from 5′-TCG to 5′-TTG or 5′-TCT) also results in a strong decrease of IFN-α production (shown in A). In contrast to IFN-α production, the secretion of IL-10 is efficiently induced by ODN with a 5′-TC lacking a 5′-CG (as shown by SEQ ID NO: 1) (shown in B). This ODN appears to be more potent for inducing IL-10 secretion than an ODN with a 5′-TTG (as shown by SEQ ID NO: 59). Therefore, ODNs that do not contain a 5′-TCG, but contain a 5′TC, efficiently induce IL-10 production from human PBMC.

### Example 5

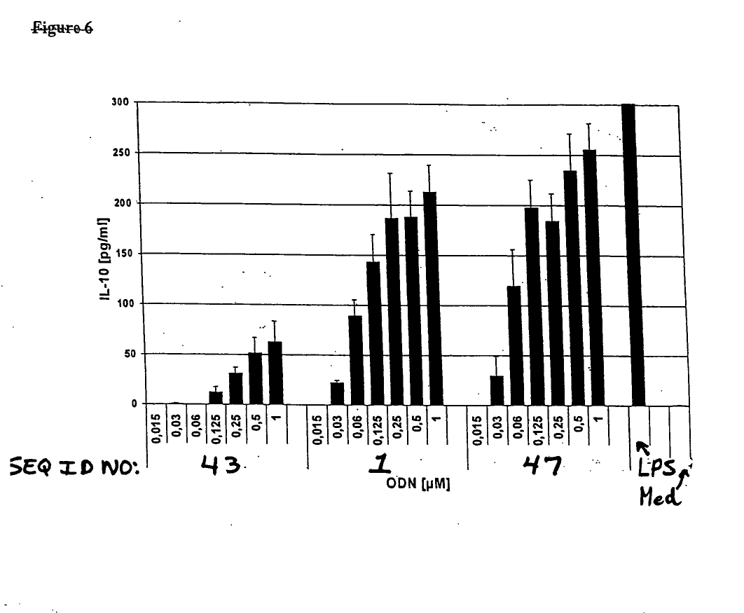

The thymidine of the 5′-TC can be chemically modified. No nucleobases other than cytosine or modifications thereof are effective in the 5′-TC dinucleotide. Human PBMC were incubated with increasing concentrations of the indicated ODN for 48 h. SN were harvested and IL-10 measured by ELISA. FIG. 5 Shows the Mean±SEM of three donors for each experimental condition.

The data demonstrate that introducing a cytosine (as in SEQ ID NO: 1) or a modified cytosine (as in SEQ ID NO: 30: 5-methyl-cytosine, and SEQ ID NO: 33: 5-hydroxy-deoxycytidine) in a thymidine-rich ODN (poly-T SEQ ID NO: 43) results in increased IL-10 amounts. This result cannot be reproduced using other bases such as guanosine or adenosine (as in SEQ ID NO: 29 or SEQ ID NO: 28). ODN with a 5′-TC, 5′-UC (U: uracile), 5′-5TC (5T: 5-methoxythymidine) all appear to have similar activities. Therefore, a cytosine or cytosine analogue is required for efficient IL-10 production.

### Example 6

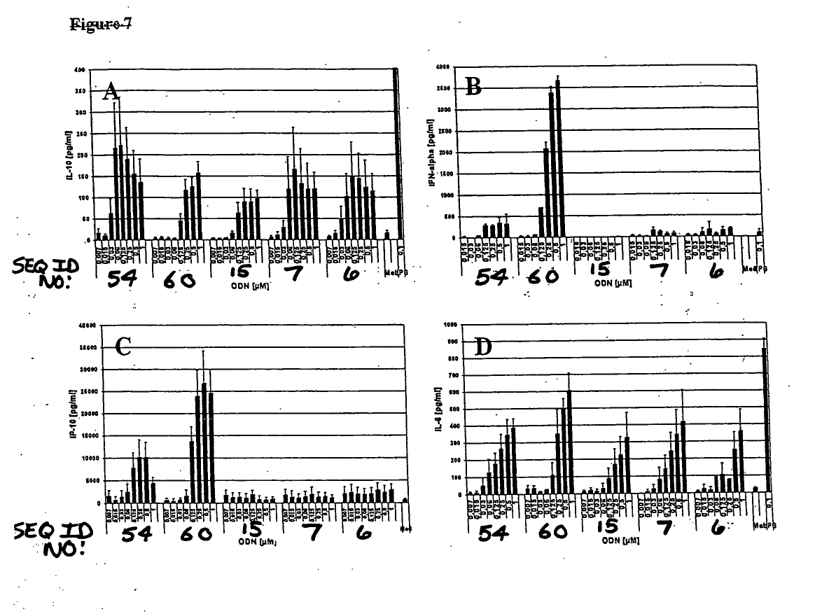

ODN with a 5′-TC as well as a 3′ shifted CpG both induce stronger IL-10 production relative to their respective ODN sequences lacking a 5′-TC or CpG. Human PBMC were incubated with increasing concentrations of the indicated ODN for 48 h. SN were harvested and IL-10 measured by ELISA. FIG. 6 shows the Mean±SEM of three donors for each experimental condition.

### Example 7

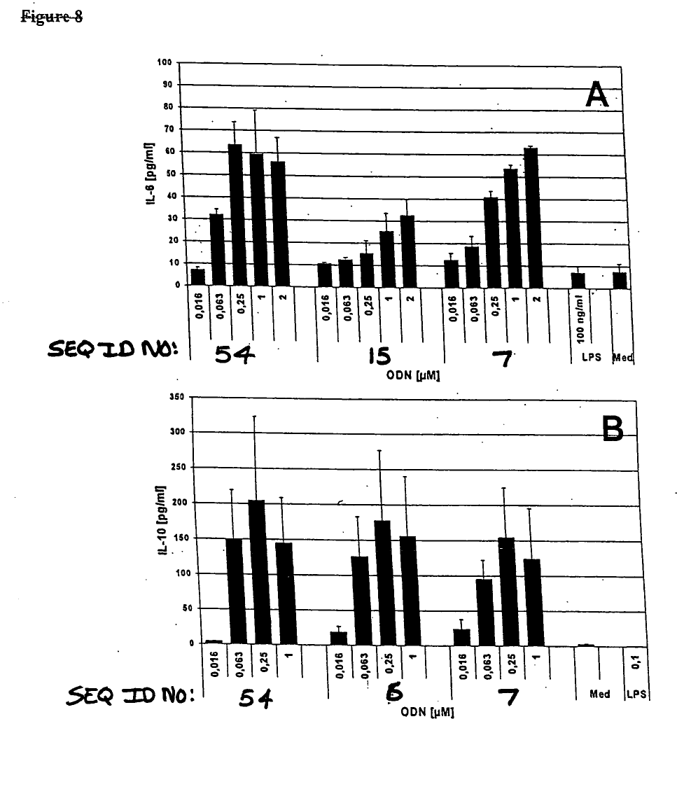

ODN with a 5′-TC as well as a 3′ shifted CpG dinucleotide induce strong secretion of IL-6 or IL-10 but show inefficient stimulation of Th1 cytokines or chemokines such as IFN-α or IP-10. Human PBMC were incubated with increasing concentrations of the indicated ODN for 48 h. SN were harvested and IL-10 (A), IFN-α (B), IP-10 (C) or IL-6 (D) measured by ELISA. FIG. 7 shows the Mean±SEM of two (B) or three donors (A, C and D).

The data demonstrate that combining a 5′-TC with a central CpG dinucleotide results in ODN with potent and efficient stimulation of a variety of cytokines such as IL-6 or IL-10. In contrast, these ODNs result in weak IFN-α and IP-10 secretion compared to the B-Class ODN SEQ ID NO: 54 and C-Class ODN SEQ ID NO: 60. These ODNs are referred to as T-Class ODNs.

### Example 8

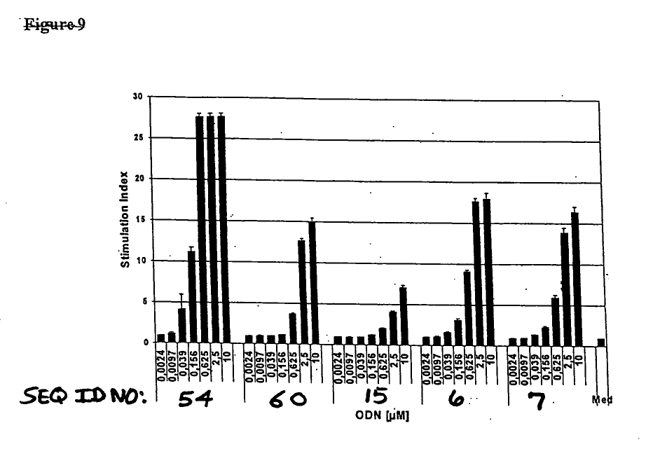

T-Class ODNs efficiently induce the production of IL-6 and IL-10 from highly purified human B cells. B cells were isolated from human PBMC and cultured with the indicated ODN for 24 h. SN were harvested and IL-6 (A) or IL-1 (B) measured by ELISA. FIG. 8 shows the Mean±SEM of two donors for each experimental condition.

The data demonstrate that the source of IL-10 or IL-6 produced upon culture of human PBMC with T-Class ODNs are B cells. Therefore, this appears to be a direct effect. Indeed, IL-10 secreting B cells were previously demonstrated to play an important role as IL-10 producers and, therefore, in Th2-biased immune responses or the induction of regulatory T or B cells.

### Example 9

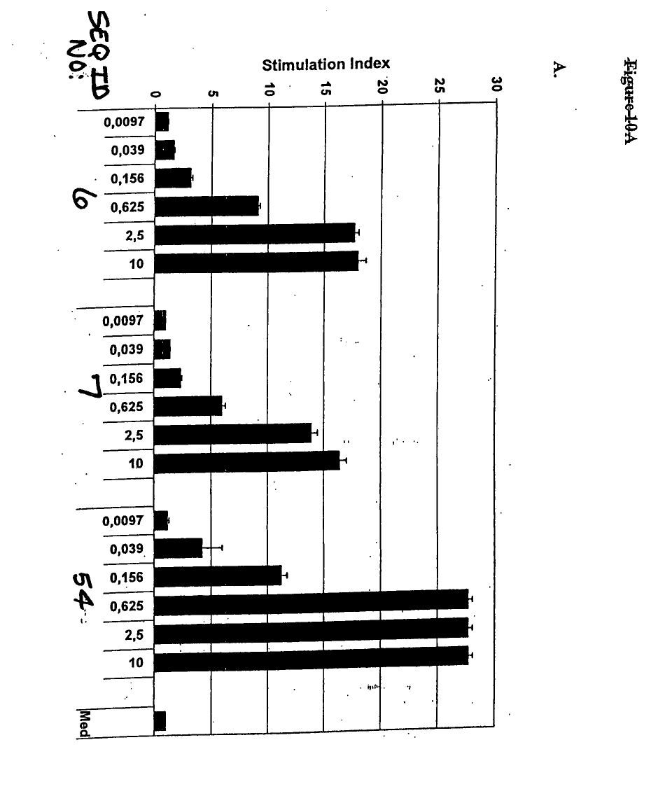

Cells expressing the human TLR9 and an NFκB-Luciferase reporter are stimulated by T-Class ODN. Transfectants expressing the human TLR9 are incubated for 16 h with the indicated ODN concentrations. Cells were lysed and NFκB stimulation was measured through luciferase activity. The results are shown in FIG. 9. Stimulation indices were calculated in reference to luciferase activity of medium without addition of ODN (fold induction of luciferase activity).

The data demonstrate that reconstitution of TLR9 expression in a non-expressing cell leads to the ability to mediate NFκB stimulation upon incubation with T-Class ODN. Therefore, the data strongly suggest that T-Class ODN stimulate the immune system via TLR9.

### Example 10

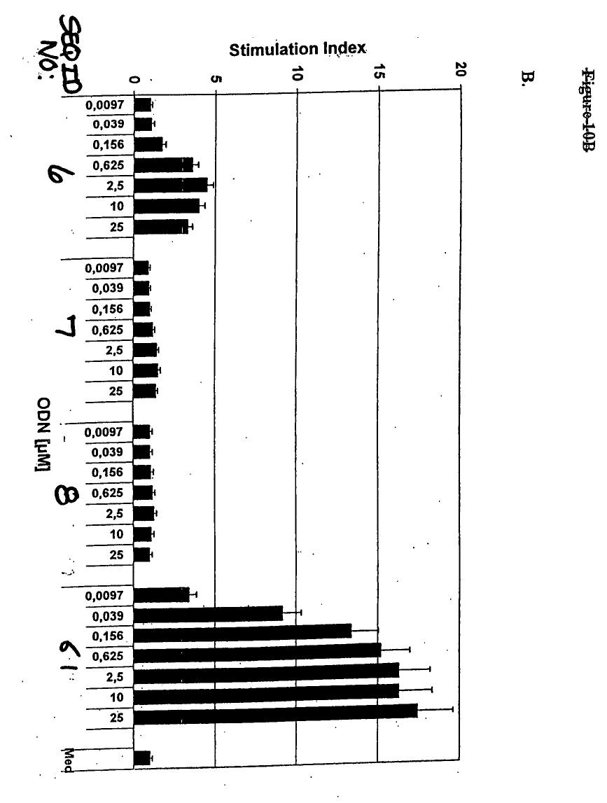

TLR9-mediated NFkB activation was measured in cells transfected with murine or human TLR9. FIG. 10 shows the results for human cells in panel A and murine cells in panel B. A surprisingly strong dependency on the position of the CpG dinucleotide was observed in the murine TLR9 transfectants relative to the human TLR9 transfectants with this class of ODN (T-Class). In these experiments, murine TLR9 did not show a significant NFkB signaling response to ODN with CpG at positions 14 (cytosine) and 15 (guanosine) or further to the 3′ end (B). In contrast, human TLR9 transfectants responded strongly to ODN with CpG at positions 14 and 15 (A). In addition, in these experiments, the T-Class ODN resulted in a more powerful stimulation in human than in murine TLR9 transfectants.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

The disclosures of all of the patents, patent applications, scientific publications, and other references are incorporated herein by reference in their entirety.

1. An oligonucleotide chosen from:
a)
5′ XYN₁YZN₂ 3′
wherein 5′ designates the 5′ end of the oligonucleotide and 3′ designates the 3′ end of the oligonucleotide, wherein X is a T or modified T nucleotide, wherein Y is a C or modified C nucleotide, wherein Z is a G or modified G nucleotide, wherein N₁ and N₂ are polynucleotides that do not include a CG dinucleotide, wherein N₁ does not include 5′ Z nucleotide, and wherein a 3′ polynucleotide consisting of the YZ dinucleotide and the N₂ polynucleotide contains a number of nucleotides that is at most 45% of the number of nucleotides in the oligonucleotide; and
b)
5′ XY N₁YZ N₂ 3′
wherein 5′ designates the 5′ end of the oligonucleotide and 3′ designates the 3′ end of the oligonucleotide, wherein X is a T or modified T nucleotide, wherein Y is a C or modified C nucleotide, wherein Z is a G or modified G nucleotide, wherein N₁ is a polynucleotide of 5 to 10 nucleotides, wherein N₁ does not include a CG dinucleotide, wherein N₁ does not include 5′ Z nucleotide, and wherein N₂ is a polynucleotide of 5 to 30 nucleotides.

2. The oligonucleotide of claim 1, wherein the oligonucleotide includes at least 1 modified internucleotide linkage.

3. The oligonucleotide of claim 1, wherein the oligonucleotide includes at least 50% modified internucleotide linkage.

4. The oligonucleotide of claim 1, wherein all internucleotide linkages of the oligonucleotide are modified.

5. The oligonucleotide of claim 1, wherein the oligonucleotide consists of 10 to 100 nucleotides.

6. The oligonucleotide of claim 2, wherein the modified internucleotide linkage is a phosphorothioate linkage.

7. The oligonucleotide of claim 2, comprising a phosphodiester linkage between a 5° C. nucleotide and a 3′ G nucleotide.

8. The oligonucleotide of claim 2, comprising a R-phosphorothioate linkage between a 5° C. nucleotide and a 3′ G nucleotide.

9-28. (canceled)

29. The oligonucleotide of claim 1 consisting of at least 55% T nucleotides.

30. A pharmaceutical composition comprising an oligonucleotide of claim 1 in combination with a therapeutic agent selected from the group consisting of chemotherapeutic agents, radiotherapeutic agents, monoclonal antibodies, and anticancer agents.

31. A method of specifically increasing IL-10 expression relative to IFN-α expression in a subject, the method comprising the step of administering an oligonucleotide of claim 1 to a subject in need of increased IL-10 expression relative to IFN-α expression.

32. The method of claim 31, wherein the ratio of induced IL-10 to IFN-α is higher than a reference ratio of IL-10 to IFN-α.

33. (canceled)

34. A method of inducing an antigen-specific regulatory T cell response in a subject, the method comprising the step of:
administering an oligonucleotide of claim 1 to a subject exposed to an antigen.

35. A method of inducing an antigen-specific regulatory B cell response in a subject, the method comprising the step of:
administering an oligonucleotide of claim 1 to a subject exposed to an antigen.

36-39. (canceled)

40. A method of treating an allergy or asthma, the method comprising the steps of:
exposing a subject to an allergen; and
administering an oligonucleotide of claim 1 to the subject, wherein the oligonucleotide is administered in an amount sufficient to prevent or alleviate an allergic response to the allergen in the subject.

41. The method of claim 40, further comprising the step of administering IL-10 to the subject.

42. A method of treating an autoimmune disease in a subject, the method comprising the steps of:
exposing a subject to a self antigen; and
administering an oligonucleotide of claim 1 to the subject, wherein the oligonucleotide is administered in an amount sufficient to prevent or treat an autoimmune disease in the subject.

43. The method of claim 42, further comprising the step of administering IL-10 to the subject.

44. A method of reducing an antigen-specific response to an implant in a subject, the method comprising the steps of:
exposing a subject to an implant antigen; and
administering an oligonucleotide of claim 1 to the subject, wherein the oligonucleotide is administered in an amount sufficient to prevent or reduce an antigen-specific response to the implant in the subject.

45. The method of claim 44, further comprising the step of administering IL-10 to the subject.

46-53. (canceled)
